SEQUENCE LISTING (47 sequences)

SEQ ID NO: 1 - protein, 16 aa, Rattus norvegicus
SQNEQDTVLDGDFIVR

SEQ ID NO: 2 - protein, 11 aa, Rattus norvegicus
IPAQGGTNINK

SEQ ID NO: 3 - DNA, 2958 bp, Rattus norvegicus
  ref <301>: AUTHORS: Soury, E., Olivier, E., Daveau, M., Hiron, M., Claeyssens, S.,Risler, J.L., and Salier, J.P.
  ref <302>: TITLE: The H4P heavy chain of inter-alpha-inhibitor family largely differs in the strucure and synthesis of its proline-rich region from rat to human
  ref <303>: JOURNAL: Biochem. Biophys. Res. Comm.
  ref <304>: VOLUME: 243
  ref <305>: ISSUE: 2
  ref <306>: PAGES: 522-530
  ref <307>: DATE: 1998-02-13
  ref <308>: DATABASE ACCESSION NUMBER: Y11283
  ref <309>: DATABASE ENTRY DATE: 1998-03-03
  ref <313>: RELEVANT RESIDUES: (1)..(2958)
acgaactggagacaaatgaagagccctgcccctgcccacatgtggaacattgtactggtcttgctctcgctgttggctgtgcttccgatcactactactgagaagaatggcatcgatatctacagtctcacagtggactcccgggtctcttcccgatttgctcatactgttgttaccagccgggtggtcaacagagccgatactgttcaagaagcgaccttccaagtagagctacccaggaaagccttcatcaccaacttctccatgatcattgatggtgtgacctacccagggttgtcaaagagaaggctgaagccagaagcaatacactgctgctgtggccggggagagagcgctggccttgtcaagaccactgggagaaagacagagcagtttgaagtgtcagtcaacgtggcccctggttccaagactaccttcgaactcatataccaagagctgctccaaagacggctgggaatgtatgagctactcctcaaagtgaggcctgagcagctggtcaagcaccttcagatgacatctacatctttgagccctcagggtatcagcaccctggagacagagagtactttcatgacccaggagttggcaaatgcccttaccacttcacagaacaagaccaaggcacatatccagttcaagccgacactctcccagcaacggaagtctcagaatgagcaggacacggtgctagatggggatttcaccgttcgctatgatgtggaccggtcttccactggcggataccttcagattgagaacggctactttgtgcaccactttgccccagaggaccttcctacaatggccaagaatgtgctctttgtcattgataaaagcggatctatggcaggcaagaaaatccaacagacccgagaagccctaatcaagatcttgaaagacctcagcacccaagaccagttcaatatcattgtgttcagtggggaagcaaaccagtgggagcagttgctggtgcaagcaacagaagagaacttgaacagggcggttgactatgcttccaagatcccggctcagggagggaccaacatcaataaagcagtgctatcggctgtggaactgctggataaaagcaaccaggctgagctactgccctccaagagcgtttccctcatcatcctgctcacggatggcgagcccactgtgggggagaccaatcccaagattatccagaagaacacacaggaagccatcaatgggcggtatagcctcttctgcctggggtttggctttgatgtgaactatcctttcctggagaagctggccctggacaacggaggcctggcccggcgcatctacgaggactcagactctgctctgcagcttcaggacttctaccaggaagtggccaatccgctgctgtcatcagtgacctttgaatatcccagcaatgctgtggaggacgtcacgcggtacaacttccaacaccactttaagggctcagagatggtggtggctgggaagctccgggaccagggccctgatgtcctcttagccaaagtcagtgggcagatgcacctgcagaacatcactttccaaacggaggccagcatagcccaacaagagaaagagttccagggtcctaagtacatctttcataactttatggagagactctgggcgttgctgaccatacagcaacagctggagcagaggatttcagcctcaggagctgagttagaggccctcgaggcccaagttctgaacttgtcactcaagtacaattttgtcactcctctcacgcacatggtggtcaccaaacctgaagatcaagaacaattccaagttgctgagaagcctacggaagtcgatggtggagtgtggagtatcctctcagcagttcaacggcatttcaagactcctaccacaggatctaaactgctgacatccaggctgagaggaaataggttccagacattgtccagactcggggatggtctcgttggatctagacaatacatgcctcctcctggacttcctggacctcctggacttcctggacctcctgggcctcccggacatcctcattttgcttctagcattgactacggcaggcagccttccttgggaagggtgctagacctgccatccttatcctcacaagatccagccggcccaagtctagccatgttaccgaaagtggtggaacaagaaggcaccacaccagaggaatccccaaacccagaccacccccgggctcctaccatcatcctgccgcttccgggatctggtgtggaccagctctgtgtggatatcttacattctgagaagcccatgaagctgtttgtagacatcaatcaggggctggaggtggttggcaagtatgagaagaatatcgggttctcatggatcgaagtgaccatcctgaagcctcacctgcaggtccatgcaacgcctgaacgactggtggtgacaaggggccgaaaaaactctgaatacaagtggaagaagacactgttctctgtgttacctggcttaaagatgaccatggataagacgggactgctacagctcagtggcccagacaaagtcaccatcagcctcttgtctctggatgaccctcagagaggactcatgctgcttttgaatgacactcatcacttctccaacgacattacaggggagcttggtcagttttaccaggatatcatctgggatgatacaaaacagacagtcagagttctaggaatcgactacccggctaccagagagctcaagttgagttatcaagacgggttcccgggaacagagatttcctgctggacggtgaagatatagaactgacaggagcattgtttgctacctgccatgttgtcctcgtatgcaggcggatgacactgtgtgccaacagggccgcctgtgaggcctagaccttgatggggaagaggatgctctcttgttacaaataaagaagggtgatgt

SEQ ID NO: 4 - protein, 932 aa, Rattus norvegicus
  ref <301>: AUTHORS: Soury, E., Olivier, E., Daveau, M., Hiron, M., Claeyssens, S.,Risler, J.L., and Salier, J.P.
  ref <302>: TITLE: The H4P heavy chain of inter-alpha-inhibitor family largely differs in the structure and synthesis of its proline-rich region from rat to human
  ref <303>: JOURNAL: Biochem. Biophys. Res. Comm.
  ref <304>: VOLUME: 243
  ref <305>: ISSUE: 2
  ref <306>: PAGES: 522-530
  ref <307>: DATE: 1998-02-13
  ref <308>: DATABASE ACCESSION NUMBER: Y11283
  ref <309>: DATABASE ENTRY DATE: 1998-03-03
  ref <313>: RELEVANT RESIDUES: (1)..(932)
MKSPAPAHMWNIVLVLLSLLAVLPITTTEKNGIDIYSLTVDSRVSSRFAHTVVTSRVVNRADTVQEATFQVELPRKAFITNFSMIIDGVTYPGLSKRRLKPEAIHCCCGRGESAGLVKTTGRKTEQFEVSVNVAPGSKTTFELIYQELLQRRLGMYELLLKVRPEQLVKHLQMTSTSLSPQGISTLETESTFMTQELANALTTSQNKTKAHIQFKPTLSQQRKSQNEQDTVLDGDFTVRYDVDRSSTGGYLQIENGYFVHHFAPEDLPTMAKNVLFVIDKSGSMAGKKIQQTREALIKILKDLSTQDQFNIIVFSGEANQWEQLLVQATEENLNRAVDYASKIPAQGGTNINKAVLSAVELLDKSNQAELLPSKSVSLIILLTDGEPTVGETNPKIIQKNTQEAINGRYSLFCLGFGFDVNYPFLEKLALDNGGLARRIYEDSDSALQLQDFYQEVANPLLSSVTFEYPSNAVEDVTRYNFQHHFKGSEMVVAGKLRDQGPDVLLAKVSGQMHLQNITFQTEASIAQQEKEFQGPKYIFHNFMERLWALLTIQQQLEQRISASGAELEALEAQVLNLSLKYNFVTPLTHMVVTKPEDQEQFQVAEKPTEVDGGVWSILSAVQRHFKTPTTGSKLLTSRLRGNRFQTLSRLGDGLVGSRQYMPPPGLPGPPGLPGPPGPPGHPHFASSIDYGRQPSLGRVLDLPSLSSQDPAGPSLAMLPKVVEQEGTTPEESPNPDHPRAPTIILPLPGSGVDQLCVDILHSEKPMKLFVDINQGLEVVGKYEKNIGFSWIEVTILKPHLQVHATPERLVVTRGRKNSEYKWKKTLFSVLPGLKMTMDKTGLLQLSGPDKVTISLLSLDDPQRGLMLLLNDTHHFSNDITGELGQFYQDIIWDDTKQTVRVLGIDYPATRELKLSYQDGFPGTEISCWTVKI

SEQ ID NO: 5 - DNA, 2963 bp, Homo sapiens
  ref <301>: AUTHORS: Tobe, T., Saguchi, K., Hashimoto, K., Miura, N.H., Tomita, M.,Li, F., Wang, Y., Minoshima, S., and Shimizu, N.
  ref <302>: TITLE: Mapping of human inter-alpha-trypsin inhibitor family heavy chain-related protein gene (ITIHL1) to human chromosome 3p21-p14
  ref <303>: JOURNAL: Cytogenet. Cell Genet.
  ref <304>: VOLUME: 71
  ref <305>: ISSUE: 3
  ref <306>: PAGES: 296-298
  ref <307>: DATE: 1995
  ref <308>: DATABASE ACCESSION NUMBER: NM_002218
  ref <309>: DATABASE ENTRY DATE: 2003-04-07
  ref <313>: RELEVANT RESIDUES: (1)..(2963)
gtgagaagcctcctggcagacactggagccacgatgaagcccccaaggcctgtccgtacctgcagcaaagttctcgtcctgctttcactgctggccatccaccagaccactactgccgaaaagaatggcatcgacatctacagcctcaccgtggactccagggtctcatcccgatttgcccacacggtcgtcaccagccgagtggtcaatagggccaatacggtacaggaggccaccttccagatggagctgcccaagaaagccttcatcaccaacttctccatgaacatcgatggcatgacctacccagggatcatcaaggagaaggctgaagcccaggcacagtacagcgcagcagtggccaagggaaagaacgctggcctcgtcaaggccaccgggagaaacatggagcagttccaggtgtcggtcagtgtggctcccaatgccaagatcacctttgagctggtctatgaggagctgctcaagcggcgtttgggggtgtacgagctgctgctgaaagtgcggccccagcagctggtcaagcacctgcagatggacattcacatcttcgagccccagggcatcagctttctggagacagagagcaccttcatgaccaaccagctggtagacgccctcaccacctggcagaataagaccaaggctcacatccggttcaagccaacactttcccagcagcaaaagtccccagagcagcaagaaacagtcctggacggcaacctcattatccgctatgatgtggaccgggccatctccgggggctccattcagatcgagaacggctactttgtacactactttgcccccgagggcctaaccacaatgcccaagaatgtggtctttgtcattgacaagagcggctccatgagtggcaggaaaatccagcagacccgggaagccctaatcaagatcctggatgacctcagccccagagaccagttcaacctcatcgtcttcagtacagaagcaactcagtggaggccatcactggtgccagcctcagccgagaacgtgaacaaggccaggagctttgctgcgggcatccaggccctgggagggaccaacatcaatgatgcaatgctgatggctgtgcagttgctggacagcagcaaccaggaggagcggctgcccgaagggagtgtctcactcatcatcctgctcaccgatggcgaccccactgtgggggagactaaccccaggagcatccagaataacgtgcgggaagctgtaagtggccggtacagcctcttctgcctgggcttcggtttcgacgtcagctatgccttcctggagaagctggcactggacaatggcggcctggcccggcgcatccatgaggactcagactctgccctgcagctccaggacttctaccaggaagtggccaacccactgctgacagcagtgaccttcgagtacccaagcaatgccgtggaggaggtcactcagaacaacttccggctcctcttcaagggctcagagatggtggtggctgggaagctccaggaccgggggcctgatgtgctcacagccacagtcagtgggaagctgcctacacagaacatcactttccaaacggagtccagtgtggcagagcaggaggcggagttccagagccccaagtatatcttccacaacttcatggagaggctctgggcatacctgactatccagcagctgctggagcaaactgtctccgcatccgacgctgatcagcaggccctccggaaccaagcgctgaatttatcacttgcctacagctttgtcacgcctctcacatctatggtagtcaccaaacccgatgaccaagagcagtctcaagttgctgagaagcccatggaaggcgaaagtagaaacaggaatgtccactcaggttccactttcttcaaatattatctccagggagcaaaaataccaaaaccagaggcttccttttctccaagaagaggatggaatagacaagctggagctgctggctcccggatgaatttcagacctggggttctcagctccaggcaacttggactcccaggacctcctgatgttcctgaccatgctgcttaccaccccttccgccgtctggccatcttgcctgcttcagcaccaccagccacctcaaatcctgatccagctgtgtctcgtgtcatgaatatgaaaatcgaagaaacaaccatgacaacccaaaccccagcccccatacaggctccctctgccatcctgccactgcctgggcagagtgtggagcggctctgtgtggaccccagacaccgccaggggccagtgaacctgctctcagaccctgagcaaggggttgaggtgactggccagtatgagagggagaaggctgggttctcatggatcgaagtgaccttcaagaaccccctggtatgggttcacgcatcccctgaacacgtggtggtgactcggaaccgaagaagctctgcgtacaagtggaaggagacgctattctcagtgatgcccggcctgaagatgaccatggacaagacgggtctcctgctgctcagtgacccagacaaagtgaccatcggcctgttgttctgggatggccgtggggaggggctccggctccttctgcgtgacactgaccgcttctccagccacgttggagggacccttggccagttttaccaggaggtgctctggggatctccagcagcatcagatgacggcagacgcacgctgagggttcagggcaatgaccactctgccaccagagagcgcaggctggattaccaggaggggcccccgggagtggagatttcctgctggtctgtggagctgtagttctgatggaaggagctgtgcccaccctgtacacttggcttccccctgcaactgcagggccgcttctggggcctggaccaccatggggaggaagagtcccactcattacaaataaagaaaggtggtgtgagcctggg

SEQ ID NO: 6 - protein, 930 aa, Homo sapiens
  ref <301>: AUTHORS: Tobe, T., Saguchi, K., Hashimoto, K., Miura, N.H., Tomita, M.,Li, F., Wang, Y., Minoshima, S., and Shimizu, N.
  ref <302>: TITLE: Mapping of human inter-alpha-trypsin inhibitor family heavy chain-related protein gene (ITIHL1) to human chromosome 3p21-p14
  ref <303>: JOURNAL: Cytogenet. Cell Genet.
  ref <304>: VOLUME: 71
  ref <305>: ISSUE: 3
  ref <306>: PAGES: 296-298
  ref <307>: DATE: 1995
  ref <308>: DATABASE ACCESSION NUMBER: NM_002218
  ref <309>: DATABASE ENTRY DATE: 2003-02-07
  ref <313>: RELEVANT RESIDUES: (1)..(930)
MKPPRPVRTCSKVLVLLSLLAIHQTTTAEKNGIDIYSLTVDSRVSSRFAHTVVTSRVVNRANTVQEATFQMELPKKAFITNFSMNIDGMTYPGIIKEKAEAQAQYSAAVAKGKNAGLVKATGRNMEQFQVSVSVAPNAKITFELVYEELLKRRLGVYELLLKVRPQQLVKHLQMDIHIFEPQGISFLETESTFMTNQLVDALTTWQNKTKAHIRFKPTLSQQQKSPEQQETVLDGNLIIRYDVDRAISGGSIQIENGYFVHYFAPEGLTTMPKNVVFVIDKSGSMSGRKIQQTREALIKILDDLSPRDQFNLIVFSTEATQWRPSLVPASAENVNKARSFAAGIQALGGTNINDAMLMAVQLLDSSNQEERLPEGSVSLIILLTDGDPTVGETNPRSIQNNVREAVSGRYSLFCLGFGFDVSYAFLEKLALDNGGLARRIHEDSDSALQLQDFYQEVANPLLTAVTFEYPSNAVEEVTQNNFRLLFKGSEMVVAGKLQDRGPDVLTATVSGKLPTQNITFQTESSVAEQEAEFQSPKYIFHNFMERLWAYLTIQQLLEQTVSASDADQQALRNQALNLSLAYSFVTPLTSMVVTKPDDQEQSQVAEKPMEGESRNRNVHSGSTFFKYYLQGAKIPKPEASFSPRRGWNRQAGAAGSRMNFRPGVLSSRQLGLPGPPDVPDHAAYHPFRRLAILPASAPPATSNPDPAVSRVMNMKIEETTMTTQTPAPIQAPSAILPLPGQSVERLCVDPRHRQGPVNLLSDPEQGVEVTGQYEREKAGFSWIEVTFKNPLVWVHASPEHVVVTRNRRSSAYKWKETLFSVMPGLKMTMDKTGLLLLSDPDKVTIGLLFWDGRGEGLRLLLRDTDRFSSHVGGTLGQFYQEVLWGSPAASDDGRRTLRVQGNDHSATRERRLDYQEGPPGVEISCWSVEL

SEQ ID NO: 7 - DNA, 25 bp, Rattus norvegicus
ggatccagtgggcagatgcacctgc

SEQ ID NO: 8 - DNA, 26 bp, Rattus norvegicus
ggatccctatatcttcaccgtccagc

SEQ ID NO: 9 - DNA, 21 bp, Rattus norvegicus
aattctgacaatcgacgccag

SEQ ID NO: 10 - DNA, 23 bp, Rattus norvegicus
gtgcttcaacattctccctcctc

SEQ ID NO: 11 - DNA, 21 bp, Rattus norvegicus
ttcccggcagcaccagtaacc

SEQ ID NO: 12 - DNA, 21 bp, Rattus norvegicus
tccctctttgcgtttggacta

SEQ ID NO: 13 - protein, 11 aa, Rattus norvegicus
ITNIPDEYFNR

SEQ ID NO: 14 - protein, 10 aa, Rattus norvegicus
NNQIDHIDEK

SEQ ID NO: 15 - protein, 9 aa, Rattus norvegicus
EEAVSASLK

SEQ ID NO: 16 - protein, 12 aa, Rattus norvegicus
SKAEAESLYQSK

SEQ ID NO: 17 - protein, 15 aa, Rattus norvegicus
TLNDMRQEYEQLIAK

SEQ ID NO: 18 - protein, 16 aa, Homo sapiens
ANTVQEATFQMELPKK

SEQ ID NO: 19 - protein, 16 aa, Homo sapiens
RVQGNDHSATRERRLD

SEQ ID NO: 20 - DNA, 22 bp, Rattus norvegicus
gcaacggaagtctcagaatgag

SEQ ID NO: 21 - DNA, 22 bp, Rattus norvegicus
gctttattgatgttggtccctc

SEQ ID NO: 22 - DNA, 21 bp, Rattus norvegicus
cttcccgatttgctcatactg

SEQ ID NO: 23 - DNA, 21 bp, Rattus norvegicus
gggctcaaagatgtagatgtc

SEQ ID NO: 24 - DNA, 21 bp, Rattus norvegicus
agggaccaacatcaataaagc

SEQ ID NO: 25 - DNA, 20 bp, Rattus norvegicus
tttggctaagaggacatcag

SEQ ID NO: 26 - DNA, 20 bp, Rattus norvegicus
tagacaatacatgcctcctc

SEQ ID NO: 27 - DNA, 19 bp, Rattus norvegicus
gtcaccaccagtcgttcag

SEQ ID NO: 28 - DNA, 20 bp, Rattus norvegicus
ggaggtggttggcaagtatg

SEQ ID NO: 29 - DNA, 18 bp, Rattus norvegicus
cgtccagcaggaaatctc

SEQ ID NO: 30 - DNA, 20 bp, Rattus norvegicus
cagacattgtccagactcgg

SEQ ID NO: 31 - DNA, 22 bp, Rattus norvegicus
agccaggtaacacagagaacag

SEQ ID NO: 32 - DNA, 18 bp, Rattus norvegicus
acaatacatgcctcctcc

SEQ ID NO: 33 - DNA, 18 bp, Rattus norvegicus
atatccacacagagctgg

SEQ ID NO: 34 - DNA, 22 bp, Rattus norvegicus
tctcagaatgagcaggacacgg

SEQ ID NO: 35 - DNA, 22 bp, Rattus norvegicus
caggcagaagaggctataccgc

SEQ ID NO: 36 - DNA, 18 bp, Rattus norvegicus
ttcaagccgacactctcc

SEQ ID NO: 37 - DNA, 20 bp, Rattus norvegicus
tttattgatgttggtccctc

SEQ ID NO: 38 - DNA, 25 bp, Rattus norvegicus
ggatccctctcagcagttcaacggc

SEQ ID NO: 39 - DNA, 26 bp, Rattus norvegicus
ggatccctatatcttcaccgtccagc

SEQ ID NO: 40 - DNA, 27 bp, Rattus norvegicus
ggatccttagccaaagtcagtgggcag

SEQ ID NO: 41 - DNA, 26 bp, Rattus norvegicus
ggatccctatatcttcaccgtccagc

SEQ ID NO: 42 - DNA, 28 bp, Rattus norvegicus
ggatccggagctgagttagaggccctcg

SEQ ID NO: 43 - DNA, 26 bp, Rattus norvegicus
ggatccctatatcttcaccgtccagc

SEQ ID NO: 44 - DNA, 30 bp, Rattus norvegicus
cgcggatccgcgtttgcttctagcattgac

SEQ ID NO: 45 - DNA, 28 bp, Rattus norvegicus
ccggaattccggctatatcttcaccgtc

SEQ ID NO: 46 - DNA, 32 bp, Rattus norvegicus
cgcggatccgcggaccagctctgtgtggatat

SEQ ID NO: 47 - DNA, 29 bp, Rattus norvegicus
cgcggatccgcgaaagtggtggaacaaga

(19) United States
(12) Patent Application Publication
(10) Pub. No.: US 2003/0232374 A1
(43) Pub. Date: Dec. 18, 2003

(54) Compositions and methods relating to detrusor estrogen-regulated protein (DERP)

(76) Inventors: George A. Kuchel, Avon, CT (US); Qing Zhu, Farmington, CT (US)

Correspondence Address: CANTOR COLBURN, LLP, 55 GRIFFIN ROAD SOUTH, BLOOMFIELD, CT, 06002

(21) Appl. No.: 10/444,575

(22) Filed: May 22, 2003

Related U.S. Application Data
(60) Provisional application No. 60/382,830, filed on May 23, 2002.

(51) Int. Cl. C12Q 1/68; G01N 33/53; G01N 33/567; C07H 21/04; C07K 14/72; C07K 16/28; C12P 21/02; C12N 5/06

(52) U.S. Cl. 435/6; 435/69.1; 435/7.2; 435/320.1; 435/325; 530/350; 530/388.22; 536/23.5

(57) ABSTRACT

Detrusor Estrogen-Regulated Protein (DERP) is involved in the pathogenesis of impaired detrusor contractility. Peptides corresponding to fragments of DERP, anti-DERP antibodies and nucleic acid primers for detection of DERP mRNA have been disclosed. Also disclosed are methods of using DERP peptides, anti-DERP antibodies and DERP nucleic acid primers. In particular, the disclosed compositions are useful for the detection of DERP and in the diagnosis and treatment of DERP-related conditions. DERP-related conditions include such conditions as impaired detrusor contractility, urinary retention, Alzheimer's disease, cardiovascular disease and osteoporosis.

### CROSS REFERENCE TO RELATED APPLICATIONS

This application claims priority from Provisional Application Ser. No. 60/382,830, filed May 23, 2002, which is incorporated herein by reference in its entirety.

### STATEMENT REGARDING FEDERALLY SPONSORED RESEARCH

The U.S. Government has certain rights in this invention pursuant to Grant No. 5P01AG004390-19 awarded by the National Institute of Health.

### BACKGROUND

The present invention relates to compositions and methods for diagnosing and treating bladder dysfunction.

Urinary incontinence (UI) refers to an involuntary leakage of urine. It is a major cause of lost independence and disability in old age, particularly among elderly women. As a result, it has a considerable impact on the well being of a growing portion of the aging population. Nearly one-third of older individuals living in the community and more than one-half of nursing home residents are incontinent. Among the latter group, 80% are women. UI has far-reaching medical and psychological implications for the affected individuals. Incontinent patients are at risk for falls, fractures, pressure ulcers, urinary infections, urosepsis as well as embarrassment, stigmatization, isolation, depression and even institutionalization.

The urinary bladder is composed of two anatomically and functionally distinct units: the detrusor (bladder body) and the bladder outlet. During the storage phase of micturition, the detrusor expands to store urine, while the outlet contracts to prevent urine outflow. In the voiding phase, the detrusor contracts and the outlet relaxes to allow for the initiation of voiding. Normal urinary function requires the coordination of the detrusor and the outlet to regulate the dual functions of storage of urine and bladder emptying at an appropriate time and place.

Recent seminal physiological studies have facilitated the understanding of bladder dysfunction in aging. In a detailed urodynamic study of 94 elderly incontinent nursing home residents, two-thirds of those studied demonstrated evidence of detrusor hyperactivity (DH) (Resnick et al., N. Engl. J. Med. 320, 1-7 (1989)). A similar proportion of the subjects studied demonstrated evidence of impaired detrusor contractility (IC). Overall, one-third of the subjects had evidence of both DH and IC leading to the characterization of the condition DHIC (Detrusor Hyperactivity with Impaired Contractility).

Detrusor biopsies from subjects with DH demonstrated an ultrastructural pattern called the dysjunction pattern. The main features of this pattern include changes in muscle structure, as well as the appearance of abnormal structures joining muscle cells, called protrusion junctions (Elbadawi et al., J. Urol. 150, 1668-80 (1993)). Protrusion junctions are long, slender processes extending from one muscle cell to a neighboring cell. It has been proposed that such changes in bladder structure and interface could facilitate propagation of heightened smooth muscle activity between muscle cells leading to involuntary detrusor contractions. Biopsies from subjects with IC, in contrast, showed a degenerative pattern with degenerative changes involving both detrusor muscle cells and also axons of nerve cells innervating the detrusor. Biopsies from subjects with DHIC showed evidence of both the dysjunction pattern and degenerative pattern. These results have clearly demonstrated the connection between DH and IC and observable changes in bladder physiology.

Normal bladder function is mediated primarily through parasympathetic and sensory fibers. The activation of parasympathetic pathways promotes bladder voiding. Acetylcholine (Ach), acting through the smooth muscle cell muscarinic Ach receptor, is the primary neurotransmitter controlling bladder voiding. Sympathetic activation promotes outlet contraction through alpha-adrenoceptors, promoting urine storage and preventing leakage. Bladder cells are thus connected both mechanically and electrically. Adherens junctions are structures which contain various members of the cadherin family of proteins and these junctions provide a mechanical means of coupling between muscle cells. Gap junctions are structures defined by the connexin family of transmembrane proteins and these junctions provide a means of electrical coupling between muscle cells. Interestingly, normal detrusor muscle cells contain adherens junctions, but gap junctions have not been observed. Detrusor muscle cells, as discussed above, contain protrusion junctions. It has been proposed that, like gap junctions, protrusion junctions may provide a means of electrical coupling between cells. Thus, the appearance of protrusion junctions in an abnormal bladder could mediate a conversion of the detrusor from a predominantly mechanical coupling (e.g., via adherens junctions) in the normal bladder to a mainly electrical coupling (e.g., via protrusion junctions) in the setting of DH. If this hypothesis is correct, then such changes in bladder structure could facilitate propagation of heightened smooth muscle activity leading to involuntary detrusor contractions.

An understanding of the causes of abnormal bladder physiology could lead to improved detection and treatment strategies for both DH and IC. Although DHIC represents the most common group of urodynamic findings in the elderly population, this problem tends to be refractory to current medical treatment. In some cases, the antispasmodic drugs used for the treatment of DH can exacerbate impaired detrusor contractility, putting the patient at risk of urinary retention leading to possible urinary tract infection, septicemia and hydronephrosis, potentially leading to renal failure. Current approaches to diagnosing DHIC all have major limitations and require sophisticated urodynamic or other invasive approaches such as repeated bladder catheterization. There thus remains a need for new, particularly simpler, more reliable and less invasive methods of treating and/or detecting disorders related to DH and IC.

### BRIEF SUMMARY

This invention relates to DERP(detrusor estrogen-regulated protein), a protein associated with impaired detrusor contractility as well as other age-related conditions. A purified immunogenic polypeptide comprises about ten to about fifty consecutive amino acids of SEQ ID NO: 4 or SEQ ID NO: 6.

In another embodiment, an isolated antibody immunochemically reactive with an immunogenic polypeptide fragment comprises about fifteen to about fifty amino acids of SEQ ID NO: 4 or SEQ ID NO: 6.

A method of detecting a DERP polypeptide in a biological sample comprises contacting the biological sample with an anti-DERP antibody and detecting any DERP polypeptide-antibody complexes formed. The method can further comprise comparing a level of DERP polypeptide-anti-DERP antibody complexes in the biological sample with a level of DERP polypeptide-anti-DERP antibody complexes in a reference sample, and diagnosing a DERP-related disorder when the level of DERP polypeptide-anti-DERP antibody complexes in the biological sample is about 25% or less of the level of DERP polypeptide-anti-DERP antibody complex in the reference sample.

A kit for the detection of DERP polypeptide in a biological sample comprises an anti-DERP antibody; a first reagent for preparation of a medium suitable for carrying out an immunological reaction; a second reagent for the detection of DERP polypeptide-anti-DERP antibody complexes formed during an immunological reaction; and a reference sample comprising a known quantity of a DERP polypeptide.

Also described is a method of detecting a DERP mRNA comprising contacting a sample with a first primer and a second primer each comprising greater than or equal to about ten nucleotides of SEQ ID NO: 5 or SEQ ID NO: 6, performing a reverse transcription and polymerase chain reaction; and detecting the reaction product; wherein detecting a reaction product confirms the presence of the DERP mRNA.

### BRIEF DESCRIPTION OF THE DRAWINGS

Referring now to the drawings wherein like elements are numbered alike in several FIGURES:

FIG. 1 shows estrogen receptor mRNA expression in the detrusor, bladder outlet and ovary as obtained by reverse transcription and polymerase chain reaction (RT-PCR) using primers for estrogen receptor alpha (ER-(α);

FIG. 2 shows estrogen receptor MRNA expression in the detrusor, bladder outlet and ovary as obtained by RT-PCR using primers for estrogen receptor beta (ER-β);

FIG. 3 shows the structure of detrusor muscle fascicles and extracellular spaces in sections obtained from sham operated female rats, wherein fascicles (F) of several muscle cells, many nucleated, as well as narrow largely empty spaces between fascicles are observed;

FIG. 4 shows the structure of detrusor muscle fascicles and extracellular spaces in sections obtained from bilaterally ovariectomized (OVx) female rats;

FIG. 5 shows the loss of detrusor muscle bulks (sarcopenia) 4 months after OVx;

FIG. 6 shows reduced nucleated detrusor muscle cells (nucleated) after OVx, wherein the number of cells is significantly lower (asterisk) after OVx (n=7), as compared to sham-operated (n=7) animals (p<0.05);

FIG. 7 shows fewer smaller muscle profiles (less than 50 μm²) in images from OVx (n=6) than in images from sham-operated animals (n=6);

FIG. 8. shows the detrusor after OVx wherein profiles of degenerated (arrows) and intact (thick arrow) axons are shown;

FIG. 9 shows a comparison of carbachol-stimulated contractile force in 12 muscle strips from 4 sham-operated and 12 muscle strips from 4 OVx animal. A. Generated tension is greater in muscle strips from sham-operated than strips from OVx animals (2-way ANOVA; p <0.001), B. Tensions expressed per muscle strip weight reduced. Differences are statistically significant (2-way ANOVA; p<0.05), C. and D. Generated tension as a percentage of maximal contraction for that strip expressed per strip (C) or strip weight (D);

FIG. 10 shows caveolar down regulation following OVx. A. Depicts detrusor tissue from a sham-operated rat; B. Depicts myocyte sarcolemma in an OVx specimen; and C. Depicts the number of caveolae per 1000 pixels of membrane perimeter in high magnification photomicrographs of detrusor tissues from 5 pairs of sham and OVx rats;

FIG. 11 shows Western blot analysis for caveolin-1 and α-smooth muscle actin proteins using equal amount of protein extract from detrusors of paired sham-operated and OVx animals;

FIG. 12 shows a graph of densitometry results comparing caveolin-1 and α-smooth muscle actin protein expression in sham and OVx animals;

FIG. 13 shows a Western blot of caveolin- 1 and α-smooth muscle actin in animals treated for 1 month with subcutaneous silastic implants containing placebo (OVx) or E2 (OVx+E2) four months after OVx surgery;

FIG. 14 shows a Western blot for caveolin-1 and α-smooth muscle actin in detrusors obtained from newborn (day 2) and young adult (one month old) female rats;

FIG. 15 shows a graph comparing caveolin-1 and α-smooth muscle actin protein expression in detrusors obtained from newborn (day 2) and young adult (one month old) female rats;

FIG. 16 shows 2-D gel protein patterns wherein the protein extracts were obtained from detrusors of sham (A), OVx (B) and OVx +E2 treated (C) animal;

FIG. 17 shows a Western blot carried out following SDS-PAGE (A) and 2-D gel electrophoresis (B) using detrusor protein extracts from sham or OVx animals and a probe of a polyclonal antibody against lumican;

FIG. 18 shows a Western blot wherein the protein samples were obtained from sham (A) or OVx (B) rats using antiserum against pig ITI-H4;

FIG. 19 shows staining of an ITI-H4 antibody in cultured rat smooth muscle cells with and without the addition of estrogen; and

FIG. 20 shows a Western blot using an anti-DERP antibody wherein the protein samples were obtained from sham and OVx samples and the antibody used was an anti-DERP antibody.

FIG. 21 shows RT-PCR of the DERP mRNA in various tissues. The legend is: -control is no RT reaction, +control is RT-PCR of liver tissue, CB is cerebellum, CTX is cortex and HPC is the hippocampus.

### DETAILED DESCRIPTION OF THE PREFERRED EMBODIMENTS

It has been discovered herein that estrogen and estrogen receptors are involved in smooth muscle function in the bladder and in the pathogenesis of impaired detrusor contractility. In particular, it has been discovered that the polypeptide Detrusor Estrogen-Regulated Protein (DERP), which is made by bladder muscle cells, is associated with impaired detrusor contractility as well as other age-related conditions. The DERP polypeptide was identified by microsequencing peptides corresponding to proteins whose levels are decreased in rats having lowered estrogen levels as a result of ovariectomy. This invention particularly encompasses the two DERP peptides identified as SEQ ID NO: 1 and SEQ ID NO: 2.

It is hypothesized that levels of the DERP polypeptide will be lower in individuals, particularly women, likely to suffer from bladder weakness (lower detrusor contractility). As used herein, a DERP-related condition is a condition in which the development or progression of the condition is related to or can be assayed by the level of the DERP polypeptide in a biological sample. Examples of DERP-related conditions include, but are not limited to, impaired detrusor contractility, urinary retention, Alzheimer's disease or related causes of dementia, cardiovascular disease and osteoporosis.

Based on the peptides identified as SEQ ID NOs: 1 and 2, a search of all available EST (expressed sequence tag) databases showed that, at least at the nucleotide and amino acid level, DERP and inter-alpha (globulin) inhibitor H4 (ITI-H4) are the same or similar. However, ESTs are often from liver tissue. Thus, DERP and ITI-H4 may differ if the non-hepatic tissues such as the bladder express a form which is different at the nucleotide/amino acid level or if there are differences in post-translational processing such as glycosylation.

ITI-H4 (and by analogy DERP) belongs to a family of inter-alpha trypsin inhibitors (the ITI family). The ITI family includes serine protease inhibitors having a highly conserved N-terminal 2/3 and a highly variable C-terminal ⅓. Proteins of this family harbor a von Willibrand type A (VWA) domain suggesting a heterophilic binding capacity, a calcium binding site, and a reactive site as α-2-thiol protease inhibitors. Analysis of the amino acid sequences reveals that ITI-H4 has several potential N-glycosylation sites. The C-terminal non-conserved region has a proline-rich domain, a bradykinin-like domain (suggesting vulnerability to cleavage), and a region homologous to the ATP-dependent proteases. DERP also contains a Vault particle-like domain. While the precise role of vault particles is not known, vault particles have been found associated with estrogen receptors leading to the hypothesis that vault domains are involved in estrogen trafficking.

Purified polypeptides comprising ITI-H4 of rat (SEQ ID NO: 4) and of human (SEQ ID NO: 6) and purified fragments thereof are included in this invention. While it is recognized that DERP may not be identical to ITI-H4, purified polypeptides and polypeptide fragments based on SEQ ID NOs: 4 and 6 are useful for compositions and methods relating to DERP.

An “isolated” or “purified” polypeptide or fragment thereof is substantially free of cellular material or other contaminating polypeptide from the cell or tissue source from which the protein is derived, or substantially free of chemical precursors or other chemicals when chemically synthesized. The language “substantially free of cellular material” includes preparations of polypeptide in which the polypeptide is separated from cellular components of the cells from which it is isolated or recombinantly produced. Thus, polypeptide that is substantially free of cellular material includes preparations of polypeptide having less than about 30%, 20%, 10%, or 5% (by dry weight) of heterologous polypeptide (also referred to herein as a “contaminating polypeptide”). Preferably, the preparation is at least about 75% by weight pure, more preferably at least about 90% by weight pure, and most preferably at least about 95% by weight pure. A substantially pure DERP polypeptide may be obtained, for example, by extraction from a natural source (e.g., an insect cell); by expression of a recombinant nucleic acid encoding a DERP polypeptide; or by chemically synthesizing the polypeptide. Purity can be measured by any appropriate method, e.g., by column chromatography, polyacrylamide gel electrophoresis, or by high pressure liquid chromatography (HPLC) analysis.

In one embodiment, there are provided DERP polypeptide fragments comprising a SEQ ID NO: 1, SEQ ID NO: 2, and mixtures thereof. In addition, also provided are polypeptides corresponding to a contiguous portion of SEQ ID) NO: 4 and/or SEQ ID NO: 6 referred to as polypeptide fragments. A polypeptide fragment corresponds to about 10 to about 50 contiguous amino acids of SEQ ID NO: 4 or SEQ ID NO: 6. Within this range, greater than or equal to about 15 contiguous amino acids is preferred, with greater than or equal to about 20 amino acids more preferred. Also within this range, less than or equal to about 30 amino acids is preferred, with less than or equal to about 25 amino acids more preferred. Preferably, the polypeptide fragment is an immunogenic fragment. As used herein, immunogenic fragment refers to a polypeptide fragment that can induce a specific immune response in appropriate animals or cells and bind with specific antibodies.

This invention also relates to polypeptides homologous to DERP. One of ordinary skill in the art can prepare a variety of polypeptides that are substantially homologous to SEQ ID NOs: 4 and 6, or allelic variants thereof, and that retain the properties of the wild-type DERP polypeptide. Substantially homologous refers to polypeptides that are greater than about 60% homologous, preferably greater than about 75% homologous and more preferably greater than about 90% homologous to a polypeptide of interest. As expressed and claimed herein the language, “a polypeptide as defined by SEQ ID NO: 4 or 6” includes all allelic variants and species orthologs of the polypeptide. When the amino acids forming the sequence are alpha-amino acids, either the L-optical isomer or the D-optical isomer can be used, with the L-isomer preferred. The term “polypeptide” as used herein includes modified sequences such as glycoproteins, and is specifically intended to cover naturally occurring polypeptides or proteins, as well as those that are recombinantly or synthetically synthesized, which occur in at least two different conformations wherein both conformations have the same or substantially the same amino acid sequence but have different three dimensional structures. Polypeptide fragments can have the same or substantially the same amino acid sequence as the naturally occurring protein.

DERP polypeptides and polypeptide fragments including mutated, truncated or deleted forms can be prepared for a variety of uses including the generation of antibodies, as reagents in diagnostic assays, as identifiers of other gene products involved in the regulation of bladder and smooth muscle function, as screening reagents useful for detecting compounds that can be used in the regulation of bladder and smooth muscle function, and as pharmaceutical agents useful for treating symptoms relating to bladder and smooth muscle function.

DERP proteins and polypeptides can also be produced by recombinant DNA methods or by synthetic chemical methods. For production of recombinant DERP proteins or polypeptides, SEQ ID NO: 3 and SEQ ID NO: 5 can be expressed in known prokaryotic or eukaryotic expression systems. Expression systems including bacterial, yeast, insect, mammalian, and the like can be used, as is known in the art.

In another embodiment, a DERP fusion polypeptide is provided. These mammalian fusion polypeptides are useful for generating antibodies against DERP amino acid sequences and for use in various assay systems. Therefore, the mammalian fusion polypeptides may be used, for example, to detect DERP expression and to provide a defense mechanism for DERP expression when desired. For example, DERP fusion polypeptides can be used to identify proteins that interact with the DERP protein and influence its function. This interaction may impart specificity to the ability of DERP to regulate other proteins, or it may increase or decrease the effect of DERP function. Identification of proteins that interact with DERP may provide a target for novel drugs. Physical methods, such as protein affinity chromatography, or library-based assays for protein-protein interactions, such as the yeast two-hybrid or phage display systems, can be used for this purpose. Such methods are well known in the art, and can be used, inter alia, as drug screens.

A DERP fusion polypeptide comprises at least two polypeptide segments fused together by means of a peptide bond. The first polypeptide segment can comprise in whole or in part the contiguous amino acids of a DERP polypeptide. Where in part, at least about 8 contiguous amino acids of the DERP polypeptides are used, with at least about 10 preferred, at least about 15 more preferred, and at least about 20 especially preferred. The first polypeptide segment can also be a full-length DERP protein. The second polypeptide segment can comprise a full-length polypeptide or a protein fragment or polypeptide that may or may not be derived from DERP. The second polypeptide segment can comprise an enzyme which will generate a detectable product, such as beta-galactosidase or other enzymes that are known in the art. Alternatively, the second polypeptide segment can include a fluorescent protein such as green fluorescent protein, HcRed (Clontech) or other fluorescent proteins known in the art. Additionally, the fusion protein can be labeled with a detectable marker, such as a radioactive maker, a fluorescent marker, a chemiluminescent marker, a biotinylated marker, and the like.

Techniques for making fusion polypeptides, either recombinantly or by covalently linking two polypeptide segments are well known. Recombinant DNA methods can be used to construct DERP fusion polypeptides, for example, by making a DNA construct that comprises DERP coding sequence in proper reading frame with nucleotides encoding the second polypeptide segment and expressing the DNA construct in a host cell. The DNA construct can be operably linked to sequences which facilitate protein production (i.e., promoters, etc.).

In addition to fusion polypeptides, DERP can be labeled in vitro by methods known in the art. DERP can be conjugated to such dyes as Texas Red, rhodamine dyes, Fluorescein (FTIC) and other dyes known in the art. Conjugation chemistries include succinimidyl ester, isothiocyanates, and maleimides. Detailed information about conjugatable dyes and conjugation chemistries can be found in the Molecular Probes Handbook of Fluorescent Probes and Research Products. Such fusion polypeptides can be used for the production of antibodies which may have greater specificity and sensitivity than those generated against short amino acid sequences. In addition, fusion polypeptides may be used to examine their ability to influence cell survival, proliferation and differentiation in tissue culture assays. Initial studies will be performed using primary detrusor smooth muscle cells and a vascular smooth muscle line (Ikari et al., J. Biol. Chem. 276, 11798-803 (2001)). The ability of fusion peptides to influence cell survival, proliferation and differentiation can be examined using endothelial cells, neurons, osteoblasts, osteoclasts, fibroblasts and the like.

The present invention also includes isolated (i.e., removed from their natural milieu) antibodies that selectively bind DERP or a mimetope thereof. As used herein, the term “selectively binds to” refers to the ability of antibodies of the present invention to preferentially bind to DERP and mimetopes thereof Binding can be measured using a variety of methods standard in the art including enzyme immunoassays (e.g., enzyme linked immunoassays (ELISA)), immunoblot assays, and the like; see, Sambrook et al., Eds., Molecular Cloning: A Laboratory Manual, 2nd ed., Cold Spring Harbor Laboratory Press, 1989, or Harlow and Lane, Eds., Using Antibodies, Cold Spring Harbor Laboratory Press, 1999.

Isolated antibodies of the present invention can include antibodies in serum, or antibodies that have been purified to varying degrees. Such antibodies may include polyclonal antibodies, monoclonal antibodies, humanized or chimeric antibodies, anti-idiotypic antibodies, single chain antibodies, Fab fragments, fragments produced from a Fab expression library, epitope-binding fragments of the above, and the like.

Antibodies that bind to DERP can be prepared from the intact polypeptide or fragments containing peptides of interest as the immunizing agent. The preparation of polyclonal antibodies is well known in the molecular biology art; see, e.g., Production of Polyclonal Antisera in Immunochemical Processes (Manson, ed.), (Humana Press 1992) and Coligan et al., Production of Polyclonal Antisera in Rabbits, Rats, Mice and Hamsters in Current Protocols in Immunology (1992). A host for preparation and/or administration of an antibody can mean a human or a vertebrate animal, including, but not limited to, dog, cat, horse, sheep, pig, goat, chicken, monkey, rat, mouse, rabbit, guinea pig, and the like.

A monoclonal antibody composition can be antibodies produced by clones of a single cell called a hybridoma that secretes or otherwise produces one kind of antibody molecule. Hybridoma cells can be formed by fusing an antibody-producing cell and a myeloma cell or other self-perpetuating cell line. Although numerous variations have been described for producing hybridoma cells, a method for the preparation of monoclonal antibodies is described by Kohler and Milstein, Nature 256, 495-497 (1975).

Briefly, monoclonal antibodies can be obtained by injecting mammals such as mice or rabbits with a composition comprising an antigen, thereby inducing in the animal antibodies having specificity for the antigen. A suspension of antibody-producing cells is then prepared (e.g., by removing the spleen and separating individual spleen cells by methods known in the art). The antibody-producing cells are treated with a transforming agent capable of producing a transformed or “immortalized” cell line. Transforming agents are known in the art and include such agents as DNA viruses (e.g., Epstein Bar Virus, SV40), RNA viruses (e.g., Moloney Murine Leukemia Virus, Rous Sarcoma Virus), myeloma cells (e.g., P3X63-AgS.653, Sp2/0-Ag14), and the like. Treatment with the transforming agent can result in production of a hybridoma by means of fusing the suspended spleen cells with, for example, mouse myeloma cells. The transformed cells are then cloned, preferably to monoclonality. The cloning is preferably performed in a medium that will support transformed cells, and not support non-transformed cells. The tissue culture medium of the cloned hybridoma is then assayed to detect the presence of secreted antibody molecules by antibody screening methods known in the art. The desired clonal cell lines are then selected.

A therapeutically useful anti-DERP antibody may be derived from a “humanized” monoclonal antibody. Humanized monoclonal antibodies are produced by transferring mouse complementarity determining regions from heavy and light variable chains of the mouse immunoglobulin into a human variable domain, then substituting human residues into the framework regions of the murine counterparts. The use of antibody components derived from humanized monoclonal antibodies obviates potential problems associated with immunogenicity of murine constant regions. Techniques for producing humanized monoclonal antibodies can be found in Jones et al., Nature 321: 522, (1986) and Singer et al., J. Immunol. 150: 2844, (1993). The antibodies can also be derived from human antibody fragments isolated from a combinatorial immunoglobulin library; see, for example, Barbas et al., Methods: A Companion to Methods in Enzymology 2, 119, (1991).

In addition, chimeric antibodies can be obtained by splicing the genes from a mouse antibody molecule with appropriate antigen specificity together with genes from a human antibody molecule of appropriate biological specificity; see, for example, Takeda et al., Nature 314: 544-546, 1985. A chimeric antibody is one in which different portions are derived from different animal species.

Anti-idiotype technology can be used to produce monoclonal antibodies that mimic an epitope. An anti-idiotypic monoclonal antibody made to a first monoclonal antibody will have a binding domain in the hypervariable region that is the “image” of the epitope bound by the first monoclonal antibody. Alternatively, techniques used to produce single chain antibodies can be used to produce single chain antibodies against DERP, as described, for example, in U.S. Pat. No. 4,946,778. Single chain antibodies are formed by linking the heavy and light chain fragments of the Fv region via an amino acid bridge, resulting in a single chain polypeptide.

Antibody fragments that recognize specific epitopes can be generated by techniques well known in the art. Such fragments include Fab fragments produced by proteolytic digestion, and Fab fragments generated by reducing disulfide bridges.

In another method, anti-DERP antibodies can be produced recombinantly using techniques known in the art. Recombinant DNA methods for producing antibodies include isolating, manipulating, and expressing the nucleic acid that codes for all or part of an immunoglobulin variable region including both the portion of the variable region comprised by the variable region of the immunoglobulin light chain and the portion of the variable region comprised by the variable region of the immunoglobulin heavy chain. Methods for isolating, manipulating and expressing the variable region coding nucleic acid in eukaryotic and prokaryotic hosts are disclosed in U.S. Pat. No. 4,714,681; Sorge et al., Mol. Cell. Biol. 4, 1730-1737 (1984); Beher et al., Science 240, 1041-1043 (1988); Skerra et al., Science 240, 1030-1041 (1988); and Orlandi et al., Proc. Natl. Acad. Sci. U.S.A. 86, 3833-3837 (1989).

A preferred method to produce anti-DERP antibodies includes (a) administering to an animal an effective amount of DERP (ranging in size from a polypeptide fragment to a full-length protein) or mimetope thereof to produce the antibodies and (b) recovering the antibodies.

Antibodies can be recovered and/or purified by methods known in the art. Suitable methods for antibody purification include purification on Protein A or Protein G beads, protein chromatography methods (e.g., diethyl-amino-ethyl (DEAE) ion exchange chromatography, ammonium sulfate precipitation), antigen affinity chromatography, and the like.

When used for immunotherapy, the antibodies, fragments thereof, or both, that bind to DERP may be unlabeled or labeled with a therapeutic agent. These agents can be coupled directly or indirectly to the antibody by techniques well known in the art, and include agents such as drugs, radioisotopes, lectins, toxins, and the like.

As used herein “anti-DERP antibody” refers to an antibody capable of complexing with DERP. Anti-DERP antibodies have a variety of potential uses that are within the scope of the present invention. For example, such antibodies can be used as tools to stain cells and detect the presence of a protein antigen; to stain tissues to determine the localization of antigens in their physiological setting; to screen expression libraries; or to recover DERP from a mixture of proteins and other contaminants. Anti-DERP antibodies can be used to detect DERP in a fluid sample. Anti-DERP antibodies can also be used in methods of diagnosing DERP-related conditions in a human. Furthermore, anti-DERP antibodies can be used to target agents that will bind with DERP and thereby reduce the activity of DERP. Targeting can be accomplished by conjugating (i.e., stably joining) such antibodies to agents using techniques known to those skilled in the art.

A diagnostic kit can comprise an antibody that recognizes and binds DERP (an anti-DERP antibody). The kit can contain a first reagent providing a medium suitable for carrying out an immunological reaction, a second reagent for the detection of DERP polypeptide-anti-DERP antibody complexes, and a reference sample containing a known amount of DERP polypeptide. Either the anti-DERP antibody or the second reagent can comprise a label suitable for the detection of DERP-anti-DERP complexes in the sample. The label can be, but is not limited to, enzymes, radiolabels, chromophores and fluorophores. Suitable second reagents include, for example, a labeled IgG molecule.

This invention further provides methods of detecting DERP and optionally quantitatively determining the concentration of DERP in a biological sample. The method comprises contacting a solid support with an excess of one or more anti-DERP antibodies which can form (preferably specifically) a complex with DERP under conditions permitting the anti-DERP antibody to attach to the surface of the solid support. The resulting solid support to which the anti-DERP antibody is attached is then contacted with a biological fluid sample so that the DERP in the biological fluid binds to the antibody and forms a DERP polypeptide-anti-DERP-antibody complex. The complex can be labeled directly or indirectly with a detectable marker. A suitable detectable marker includes, for example, an IgG antibody labeled for UV, fluorescent or chemiluminescent detection. Suitable detectable markers include, but are not limited to, an enzyme, biotin, a fluorophore, a chromophore, a heavy metal, a paramagnetic isotope, a radioisotope, and the like.

The amount of DERP polypeptide-anti-DERP-antibody complex formed can be quantitated thereby detecting and quantitatively determining the concentration of DERP in the biological fluid sample. Quantification can involve, for example, comparison of a signal obtained for a biological sample with one or more samples containing known quantities of DERP polypeptide.

In accordance with methods of detecting DERP polypeptide in a sample, the sample can be a cell, a tissue, or a biological fluid. Suitable biological fluids include, but are not limited to, tissue extract, urine, blood, serum, plasma, central nervous system fluid, and phlegm.

A method of diagnosing a DERP-related condition in a mammal, comprises contacting a biological sample from the mammal with an anti-DERP antibody, detecting DERP polypeptide-anti-DERP antibody complexes, and comparing a level of DERP polypeptide-anti-DERP antibody complexes in the sample with a level of DERP polypeptide-anti-DERP antibody complexes in a reference sample. A level of DERP polypeptide-anti-DERP antibody complexes in the biological sample from a mammal that is about 25% or less, preferably 10% or less, of the level of DERP polypeptide-anti-DERP antibody complex in the reference sample can produce a diagnosis of a DERP-related disorder. A suitable reference sample, for example, has a concentration of DERP polypeptide equal to or approximately equal to the amount of DERP polypeptide found in a sample from a person having normal bladder function.

Detection of DERP polypeptide in a sample can be used to diagnose the presence of impaired detrusor contractility in older individuals in a non-invasive fashion. Similarly detection of DERP in a sample can also be used to identify those individuals at risk of developing urinary retention with related complications in the future. Identification of such individuals has the potential to allow treatment prior to the development of symptoms of urinary retention. A lower level of DERP polypeptide in a biological fluid of a patient sample relative to a normal sample can indicate the presence of impaired detrusor contractility in the patient. A lower level of DERP polypeptide as used herein means less than about 25% of normal levels, preferably less than about 10% of normal levels. Detection of DERP polypeptide in a sample can, for example, be in the form of a kit using DERP antibodies in an ELISA assay.

An alternate method of determining the level of DERP in a sample is to determine the level of DERP mRNA in the sample. One method to determine the level of DERP in a sample is to use RT-PCR (reverse transcription and polymerase chain reaction) methods with primers based on either the rat or human DERP DNA sequence. RT-PCR techniques are well known in the art. The similarity of DERP to ITI-H4 allows the use of primers directed to ITI-H4 to detect DERP. Thus, primers for the DERP mRNA can be derived from the rat ITI-H4 gene (SEQ ID NO: 3, accession number NM_{—}2292982) or the human ITI-H4 gene (SEQ ID NO: 5, accession number NM_{—}002218), for example. Primers to the rat DERP include SEQ ID NO: 7 and SEQ ID NO: 8. Suitable primer include greater than or equal to about 10, preferably greater than or equal to about 15 contiguous nucleotides of SEQ ID NO: 3 or SEQ ID NO: 5.

Detection of DERP in a sample can be used to identify those individuals most likely to benefit from estrogen replacement. Estrogen replacement may provide primary, secondary or tertiary prevention of impaired detrusor contractility. Estrogen replacement could be provided in the form of 17β-estradiol (E2), as conjugated estrogens (e.g. Premarin®), as dietary estrogens (e.g., isoflavones) or as one of the newly developed SERMs—selective estrogen receptor modulators (e.g., raloxifene —Evista®). A lower level of DERP in a biological fluid of a patient sample relative to a normal sample can indicate that a patient may benefit from estrogen replacement. Detection of DERP in a sample can, for example, be in the form of a kit using DERP antibodies in an ELISA assay. In another embodiment, detection of DERP in a sample can be used to identify elderly individuals in whom antispasmodic drugs could be used with a lesser risk of developing urinary retention and its related complications. Antispasmodic drugs such as oxybutynin (Ditropan®), tolterodine (Detrol®) or amitriptyline (Elavil®) are prescribed to improve the symptoms relating to bladder instability and urgency. In some cases, particularly in the case of individuals with DHIC, antispasmodic drugs can worsen already impaired detrusor contractility and produce urinary retention which can on occasion be severe enough to require the insertion of a bladder catheter. A normal level of DERP in a patient sample may indicate that a patient could benefit from the use of antispasmodic drugs with a reduced risk of side effects. A normal level of DERP is within about 50% of a control normal sample. Detection of DERP in a sample can, for example, be in the form of a kit using DERP antibodies in an ELISA assay.

Methods for the treatment of impaired detrusor contractility are also provided. In particular, a method for the prevention or treatment of impaired detrusor contractility is provided. This method includes the use of hormone replacement therapy. As used herein, the term “hormone replacement therapy” means a treatment of a human female having reduced levels of endogenous estrogen in which a mammalian estrogen is administered to the female. The mammalian estrogen can be administered alone or in combination with at least one other compound, where the other compound is administered to inhibit the estrogen's tissue proliferative effects in the breast or uterus. The term “mammalian estrogen” refers to a hormonal steroid endogenous in mammals or other synthetic analogs that produce an estrogenic response at cellular estrogen receptors.

The mammalian estrogen can be administered to a patient having a reduced level of DERP in a therapeutically effective amount to inhibit or prevent the development of impaired detrusor contractility. A therapeutically effective amount is about 0.02 milligrams (mg)/day to about 5 mg/day of mammalian estrogen. Within this range, an amount of greater than or equal to about 0.1 mg/day is preferred, with greater than or equal to about 0.25 mg/day ore preferred. Also within this range, less than or equal to about to about 2 mg/day is preferred, with less than or equal to about 1 mg/day more preferred. Dosages of estrogen can vary depending on both the type of estrogen as well as the condition being treated (Prestwood et al., J. Clin. Endocrinol Metab. 85, 4462-9 (2000)).

Although it is preferred that the mammalian estrogen be administered on a daily basis, a therapeutically effective amount may be administered on a non-daily periodic basis, where the target dosage can be determined based upon the therapeutically effective daily dosages set forth above.

Alzheimer's Disease (AD) is the most common neurodegenerative disorder of aging, and is characterized by progressive dementia and personality dysfunction. The abnormal accumulation of amyloid plaques in the vicinity of degenerating neurons and reactive astrocytes is a pathological characteristic of AD. Alzheimer's-type dementia is thought to be due to a degenerative process, with a large loss of cells from the cerebral cortex, hippocampus and other brain areas. Acetylcholine-transmitting neurons and their target nerve cells are particularly affected. The brain shows marked atrophy with wide sulci and dilated ventricles. Senile plaques and neurofibrillary tangles are present. Memory loss is the most prominent early symptom. The signs and symptoms of Alzheimer's-type dementia can also include depression, paranoia, anxiety or any of several other psychological symptoms. The most common clinical picture is slow disintegration of personality and intellect due to impaired insight and judgment and loss of affect. Memory impairment increases beginning with problems recalling recent events or finding names. The impairment varies greatly from time to time and often from moment to moment.

DERP is also present in cells in areas of the brain that are critical in memory and in the development of Alzheimer's disease. In particular, it has been shown that the DERP mRNA is expressed in the rat hippocampus and cortex. Cells in the hippocampus are thought to be involved in the progression of Alzheimer's disease. Without being held to theory, it is hypothesized that DERP has a role in the neurodegeneration associated with aging and the progression of Alzheimer's disease.

Detection of DERP in a sample can be associated with the diagnosis of Alzheimer's disease, particularly in older women. A lower level of DERP in a biological fluid of a patient sample such as a serum or cerebrospinal fluid sample relative to a normal sample could indicate the presence of Alzheimer's disease. Detection of DERP in a sample can, for example, be in the form of a kit using DERP antibodies in an ELISA assay.

Detection of DERP in a sample can be used, for example, to identify older women with Alzheimer's disease likely to benefit from estrogen replacement. A lower level of DERP in a biological sample of a person with Alzheimer's disease relative to a normal sample can be used to identify those persons with Alzheimer's disease likely to benefit from hormone replacement therapy. Detection of DERP in a sample can, for example, be in the form of a kit using DERP antibodies in an ELISA assay. Alternatively, detection of DERP can be in the form of a nucleic acid-based method such as reverse transcription-PCR using the DERP mRNA.

Another method comprises detecting a lower level of DERP in a biological sample from a mammalian subject, diagnosing Alzheimer's disease in the subject, and treating the subject with hormone replacement therapy. Detection of DERP in a sample can, for example, be in the form of a kit using anti-DERP antibodies in an ELISA assay. Alternatively, detection of DERP can be in the form of a nucleic acid method such as reverse transcription-PCR using the DERP mRNA. Diagnosing Alzheimer's disease can be done by detecting DERP in a sample or by other methods known in the art. Hormnone replacement therapy can be performed by the previously described methods.

Cardiovascular disease is the leading cause of death in women. Compared to men, premenopausal women are relatively protected from cardiovascular disease by estrogen, but gradually lose this protection following menopause as estrogen levels decline (Lemer & Kannel, Amer. Heart J., 111: 383-90 (1986)). The onset of cardiovascular disease is hastened in women by prematurely induced surgical menopause and its attendant reduction in endogenous estrogen levels (Parrish, H. M. et al., Amer. J. Obst. Gynecol., 99: 155-62 (1967)).

Both the DERP protein and DERP mRNA are expressed in vascular smooth muscle cells. DERP could thus be a marker for cardiovascular disease, similarly to CRP (C-reactive protein). It is also hypothesized that DERP has a role in the pathogenesis of cardiovascular disease.

Detection of DERP in a sample can be associated with the diagnosis of cardiovascular disease, particularly in older women. A lower level of DERP in a biological sample of a patient sample such as a serum sample relative to a normal sample can indicate the presence of cardiovascular disease. In contrast, elevated CRP levels indicate the presence of cardiovascular disease. Detection of DERP in a sample can, for example, be in the form of a kit using DERP antibodies in an ELISA assay.

Detection of DERP in a sample can also be used to identify older women with cardiovascular disease likely to benefit from estrogen replacement. A lower level of DERP in a biological fluid of a person with cardiovascular disease relative to a normal sample can be used to identify those persons with cardiovascular disease likely to benefit from hormone replacement therapy. Detection of DERP in a sample can, for example, be in the form of a kit using DERP antibodies in an ELISA assay. Alternatively, detection of DERP can be in the form of a nucleic acid method such as reverse transcription-PCR using the DERP mRNA.

Another method comprises detecting a lower level of DERP in a biological sample from a mammalian subject, diagnosing cardiovascular disease in the subject and treating the subject with hormone replacement therapy. Detection of DERP in a sample can, for example, be in the form of a kit using DERP antibodies in an ELISA assay. Alternatively, detection of DERP can be in the form of a nucleic acid method such as reverse transcription-PCR using the DERP mRNA. Diagnosing cardiovascular disease can be done using detection of DERP in a sample or by other methods known in the art. Hormone replacement therapy can be done by the previously described methods.

Osteoporosis is characterized by low bone mass and a disruption of bone architecture that leads to an increased risk of fracture. It occurs in both men and women, but most commonly among women following menopause when the rate of bone resorption becomes greater than that of bone formation. These changes result in progressive bone loss and lead to osteoporosis in a significant proportion of women over age 50. It is estimated that 40% of 50-year-old women will sustain one or more osteoporosis-related fractures of the spine, hip or wrist during their lifetime.

Detection of DERP in a sample can be used to identify older women with osteoporosis likely to benefit from estrogen replacement. A lower level of DERP in a biological sample of a person with osteoporosis relative to a normal sample can be used to identify those persons with cardiovascular disease likely to benefit from hormone replacement therapy. Detection of DERP in a sample can, for example, be in the form of a kit using DERP antibodies in an ELISA assay. Alternatively, detection of DERP can be in the form of a nucleic acid method such as reverse transcription-PCR using the DERP MRNA.

Another method comprises detecting a lower level of DERP in a biological sample from a mammalian subject, diagnosing osteoporosis in the subject and treating the subject with hormone replacement therapy. Detection of DERP in a sample can, for example, be in the form of a kit using DERP antibodies in an ELISA assay. Alternatively, detection of DERP can be in the form of a nucleic acid method such as reverse transcription-PCR using the DERP mRNA. Diagnosing osteoporosis can be done using detection of DERP in a sample or by other methods known in the art. Hormone replacement therapy can be done by the previously described methods.

DERP can be used a marker for cell survival or differentiation as the presence of vonWillibrand domains in ITI-H4, as well as the demonstrated ability of other members of this protein family to bind with hyaluronic acid, both raise the possibility that DERP could be involved in cell survival or differentiation by a variety of cells including smooth muscle cells, endothelial cells, neurons, glial cells, osteoblasts, osteoclasts and fibroblasts.

It has been known for some time that hyaluronic acid can influence cellular survival through receptors such as CD44, yet more recent studies indicate that hyaluronic acid can also be taken up into cells and can exert a variety of important cellular effects through several types of intracellular receptors (Tammi et al., J. Biol. Chem. 277, 4581-4 (2002)). Evidence exists that other members of the inter-alpha-trypsin inhibitor family of proteins can bind to hyaluronic acid through both covalent and non-covalent mechanisms. As a result of these considerations, it is hypothesized that a similar relationship may exist between DERP and hyaluronic acid. A fluorescent conjugate of hyaluronic acid together with anti-DERP antibodies can be used to determine if cellular and extracellular co-localization exists.

Pharmaceutical compositions comprising anti-DERP antibodies, DERP polypeptides, DERP polypeptide fragments, estrogen and the like may be formulated using one or more physiologically acceptable carriers or excipients. Thus, the compounds and their physiologically acceptable salts and solvates may be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups, or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring, and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch. The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing, and/or dispersing agents. Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides. In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

The invention is further illustrated by the following non-limiting examples.

### EXAMPLE 1

### Expression of Estrogen Receptors in the Bladder

Frozen tissue from rat detrusor, bladder outlet, and ovary was ground in liquid nitrogen to a fine powder. RNA extraction was then performed by techniques known in the art. A suitable procedure is using the “RNAwiz” kit (Ambion). RNA quantification was then performed by measuring the optical density at 260 nm and 280 nm and taking the 260/280 ratio. Aliquots of the RNA were examined by agarose gel electrophoresis to determine that there was no significant degradation. A total of 2 micrograms of RNA was used for reverse transcription and polymerase chain reaction (RT-PCR) by techniques known in the art. A suitable method for reverse transcription is to use the Superscript kit (Gibco-BRL) with an oligo(dT) primer. This method allows the isolation of DNAs corresponding to poly(AA) mRNA. PCR was performed using primers specific for ER-α, ER-β, and ITI-H4 cDNAs. Table 1 shows the sequences of the PCR primers used. The ER-(α primers are nucleotides 472-492 and 794-816 of the ER-α mRNA. The ER-β primers are nucleotides 38-58 and 279-300 of the ER-β mRNA. The ITI-H4 primers are nucleotides 1986-2005 and 2432-2450 of the ITI-H4 mRNA.

TABLE 1

Reverse Transcription/PCR Primers For Estrogen Receptor Amplification:

| | | | |
|---|---|---|---|
| mRNA | SEQ ID NO | Primers | PCR program |

| | | | | |
|---|---|---|---|---|
| ER-α | 9 | 5′AATTCTGACAATCGACGCCAG3′ | Denature: | 95 ° C., 1 min |
| | 10 | 5′GTGCTTCAACATTCTCCCTCCTC3′ | Annealing: | 57 ° C., 1 min |
| | | | Extension: | 72 ° C., 1 min |
| | | | Cycles: | 30 |
| ER-β | 11 | 5′TTCCCGGCAGCACCAGTAACC3′ | Denature: | 95 ° C., 1 min |
| | 12 | 5′TCCCTCTTTGCGTTTGGACTA3′ | Annealing: | 57 ° C., 1 min |
| | | | Extension: | 72 ° C., 1 min |
| | | | Cycles: | 30 |
| ITI-H4 | 26 | 5′TAGACAATACATGCCTCCTC3′ | Denature: | 94 ° C., 1 min |
| | 27 | 5′GTCACCACCAGTCGTTCAG3′ | Annealing: | 55 ° C., 1 min |
| | | | Extension: | 72 ° C., 1 min |
| | | | Cycles: | 35 |

The PCR reaction (50 μl) contained 25 mM MgCl₂, 10 μM each of the primers and 1 unit of Taq DNA polymerase. The PCR steps were carried out as described in Table 1, except that they were concluded by a single extension step at 72° C. for 10 minutes. The PCR products were resolved and analyzed using ethidium bromide/1% agarose gel electrophoresis.

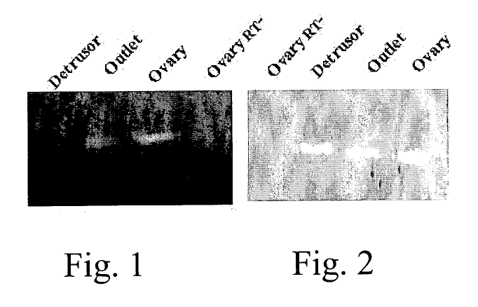

As shown in FIGS. 1 and 2, ER-α and ER-β are both abundantly expressed in ovary. The expression of ER-α is evident in the bladder outlet, yet is barely evident in the detrusor (FIG. 1). In contrast, ER-β mRNA is significantly expressed in both the detrusor and the outlet (FIG. 2). These results clearly demonstrate that the two subtypes of estrogen are differentially expressed in the bladder. These differences in expression and distribution could explain the diversity and complexity of estrogen function.

### EXAMPLE 2

### Generation of OVx Rats and E2 Treatment

Ovariectomy (OVx) of laboratory animals provides a model for the effects of estrogen levels on female physiology. In particular, OVx of mature rats can serve as a model for studies of the role of ovarian hormones, such as estrogen, on the physiology and structural integrity of the bladder, the brain, bone and cardiovascular system.

Female Fischer 344 rats were obtained at the age of 9-10 months from a single colony (Harlan Sprague Dawley, Inc., Indianapolis, Ind.). The animals were housed 2 per cage with food and water provided ad lib. Vaginal examinations and cytological studies were conducted prior to the surgery to standardize timing of the menstrual cycle. Under anesthesia with pentobarbital, either sham or bilateral ovariectomy (OVx) surgery was performed using a dorsal surgical approach. Vaginal observation and vaginal smears were performed post-surgery and one week prior to sacrificing (4 months after surgery). The presence of mature cells on vaginal cytology in OVx animals was indicative of estrogen stimulation from residual ovarian tissue and such animals were excluded from further studies. Most sham animals were in a state of irregular cycling at this age, while a few demonstrated a constant estrus pattern. Efforts were made to sacrifice animals at the stage of proestrus whenever possible.

Upon sacrificing of the rat under deep anesthesia, the bladder detrusor was immediately removed at the level of the ureter, and then cut sagitally while maintained cold.

Some animals underwent 17β-estradiol (E2) treatment before sacrificing. Silastic capsules containing E2 (for OVx rats) or placebo (for sham or OVx rats) were implanted subcutaneously on the back of the neck (Legan et al., Endocrinology 96, 50-6 (1975)).

All of the OVx animals studied demonstrated both evidence of mucosal atrophy on visual inspection, as well as complete absence of mature epithelial cells in vaginal cytological smears. In addition, during a post-mortem examination, no residual ovarian tissue was observed in any of the OVx animals, while obvious uterine atrophy was seen in all. In contrast, cytological smears from most sham-operated animals exhibit constant diestrus, a pattern typical for animals of this age. As expected, following OVx, animal body weight increased, while uterine weights decreased.

### EXAMPLE 3

### Long-term Ovariectomy Results in Sarcopenia of Detrusor

Bladder samples were prepared for electron microscopy (EM) (Elbadawi et al., J. Urol. 150, 1650-56 (1993)). Briefly, hemi-bladders were immersed overnight in freshly made pre-chilled 2.5% glutaraldehyde at 4° C., and subsequently transferred to pre-chilled 0.2M cacodylate buffer. Samples were stored refrigerated with buffer replaced weekly until trimmed. Two tissue bocks (about 1 mm³), trimmed to the size appropriate for EM, were obtained from the dome region of detrusor specimens. The blocks underwent standard processing by osmication, dehydration and embedment in Araldite. Semi-thin (1 micrometer thick) sections stained with Toluidine blue were examined by light microscopy for orientation of smooth muscle profiles. Ultrathin sections were obtained from the trimmed block that best represented the specimen and had optimal orientation. The sections were then mounted on uncoated 150 mesh grids (5 to 10 sections per grid) and stained by a uranyl nitrate/lead citrate sequence. At least two grids from each of the 2 blocks were studied in each case.

High resolution electron microscopy (EM) studies can be performed as follows. Detrusor tissues from 7 pairs of sham/OVx animals were fully processed for electron microscopic analysis. Under a JEOL JEM 100SX or a Philips EM300 electron microscope, structure of the 3 compartments: muscle cells, interstitium and intrinsic nerves of the detrusor were examined in detail and amply documented photographically (average of 90 photographs per specimen). The specific features evaluated qualitatively in each of the 3 compartments have been described previously. All EM photographs used in qualitative studies were scanned by a scanner (SnapScan 310, Agfa). Using low (4,000×) magnification images a “blinded” individual manually delineated the boundaries of individual nucleated muscle cells using image analysis software (Media Cybernetics Inc.). Only those muscle cell profiles with a whole perimeter and nucleus were traced and analyzed. The length of the muscle cell perimeter and area were measure in pixels and these values were subsequently converted to micrometers and square micrometers using internal standards. All samples and images were coded until the completion of analysis.

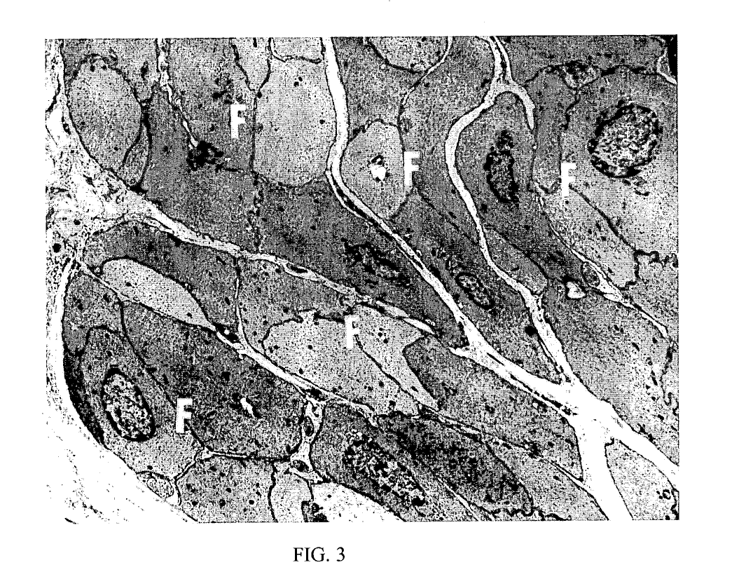

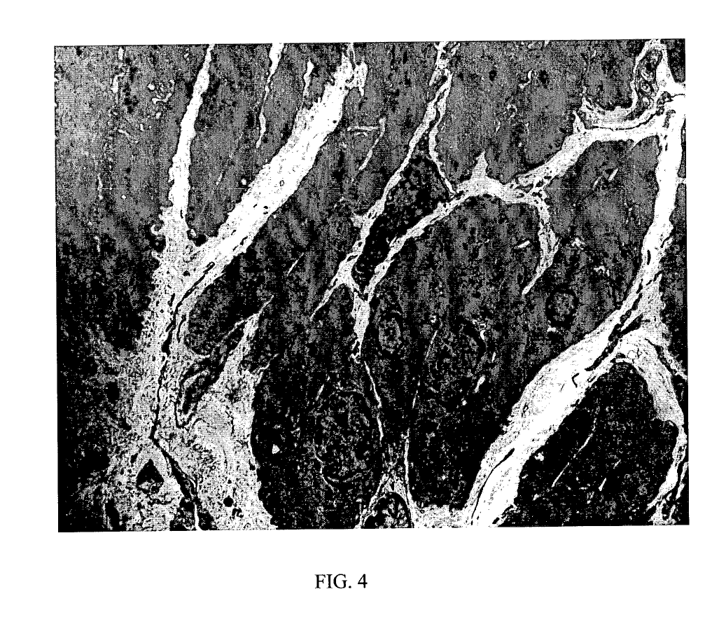

Low magnification EM images did not reveal any striking changes in the detrusor four months after ovariectomy. Muscle fascicles and cells appeared to be largely intact in both sham operated (FIG. 3) and ovariectomized (FIG. 4) animals. In the latter, the smooth muscle compartment appeared to be decreased, with possible widening of spaces between muscle fascicles. Adherens junctions joining detrusor muscle cells were visible at high magnification in both sham and ovariectomized tissues, but seemed fewer in the latter. No ultraclose abutments were observed in either group, but a single structure resembling a protrusion junction was seen in a single field of an ovariectomized animal.

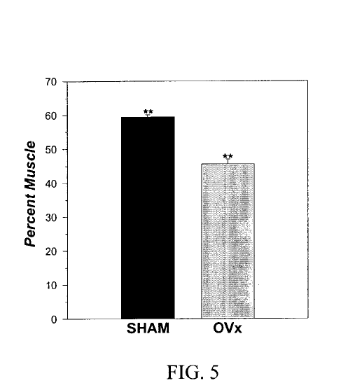

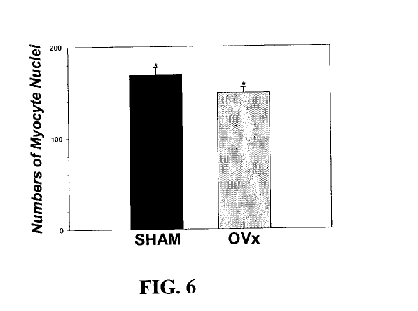

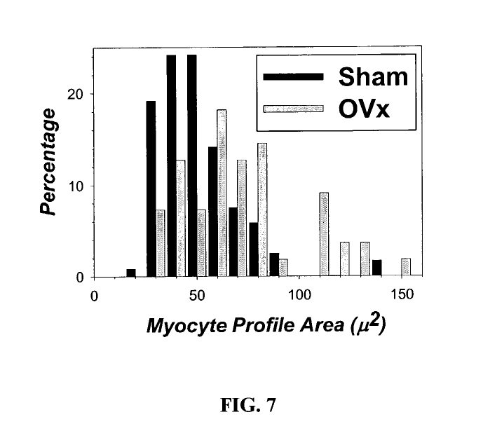

Smooth muscle represented 130,816±1957 pixels in images obtained from sham-operated animals (59.1% of the field; FIG. 5), but only 103,888±1897 (47.0%, p<0.001) in equal numbers of images form OVx animals. Counts of myocyte profiles were somewhat lower (149±6) in specimens from OVx tissues than those from sham-operated animals (169±8, FIG. 6). In low magnification EM images from OVx animals (FIG. 7), there appeared to be fewer small-sized myocyte profiles (<50 μm²) than in images from sham-operated animals, with an apparent shift towards medium-sized (50-100 μm²) and large-sized (>100 μm²) myocyte profiles.

### EXAMPLE 4

### Ovariectomy Also Results in Degeneration of Detrusor Axon Profiles

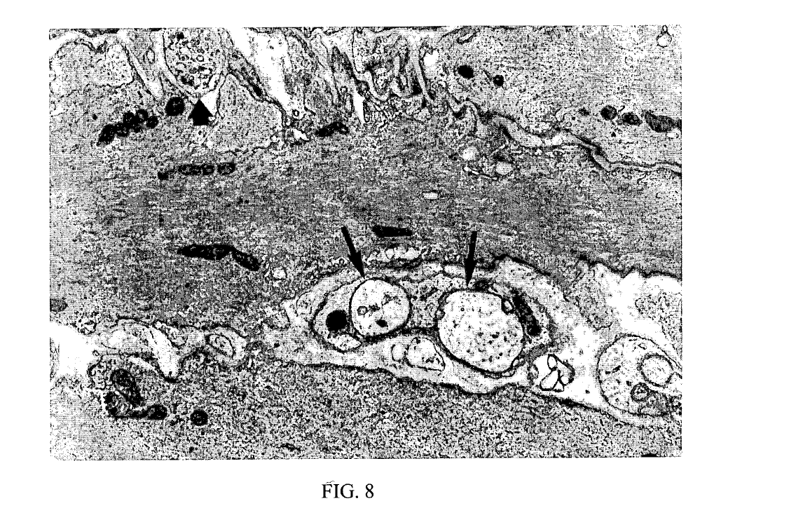

Axonal profiles were generally intact with no evidence of degeneration in detrusors from shame-operated rats (data not shown). Degeneration was observed in some intrinsic axons in OVx detrusors (FIG. 8). The degeneration was characterized by depleted synaptic vesicles, disrupted mitochondria, axoplasmic multivesicular electron-dense bodies, and disrupted axolemma with fragmentation or lysis of some profiles. The identity of axonal profiles undergoing degenerative changes could not be determined since no secretory vesicles or myelination was observed in any. Neither axon sprouts nor regenerated axon terminals were observed in any specimen. Among intact axons observed in OVx bladders were both myelinated and nonmyelinated profiles.

### EXAMPLE 5

### Detrusor Muscle Strips From OVx Rats Generate Less Tension In Response to Carbachol

Muscle strip studies can be performed by the method of Longhurst et al., J. Urol. 148, 915-919 (1992). Upon removal, the entire detrusor was placed in oxygenated (95% oxygen (O₂), 5% carbon dioxide (CO₂)) 4° C. Tyrode solution (sodium chloride (NaCl) 135 millimolar (mM); potassium chloride (KCl) 2.8 mM; calcium chloride (CaCl₂) 1.9 mM; magnesium chloride (MgCl₂) 0.4 mM; sodium phosphoric acid (NaH₂PO₄) 0.4 mM; sodium carbonate (NaHCO₃) 1.3 mM and Dextrose 6 mM). Muscle strips measuring 50 millimeters (mm)×2 mm (8-15 mg) were prepared. A total of 2 to 4 strips were cut longitudinally from each detrusor in a standardized fashion. The carbachol-stimulated contractility was compared using muscle strips obtained from sham and OVx rats, each group having 12 muscle strips from 4 animals. Each strip was suspended between paired hooks and placed in a 5 ml organ baths containing warm (31° C.) oxygenated Tyrode solution. The strips were connected to a Grass force displacement transducer (FTO3C) and an initial tension of 1 gram was applied during an equilibration period of 60-90 minutes. Responses were recorded on a Grass Polygraph (Model 7). Concentration-response curves were measured by applying increasing concentrations of Carbachol (Sigma; St. Louis, Mo.) and waiting for the response to plateau. The interval between applications increased from 15-30 minutes as higher doses were reached. The addition of each concentration of carbachol was followed by several washes with drug-free buffer. Muscle strips were then blotted dry and weighed.

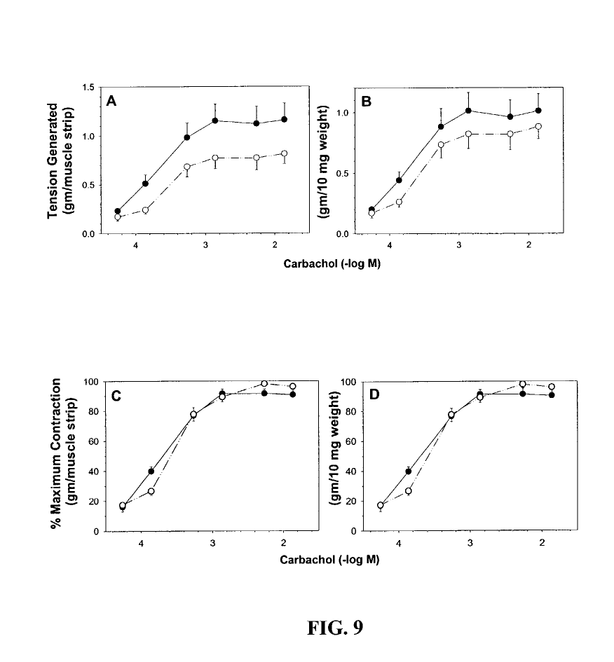

The tension generated at different carbachol concentrations by detrusor strips from OVx rats was 30-50% less than that by strips of the same length from sham-operated animals (FIG. 9A; p<0.001). Although cut to the same length, strips obtained from OVx animals tended to weigh less (9.3±0.8 mg vs. 11.4±0.7 mg, NS). When the tension generated was corrected for strip weight, the differences in tension became considerably smaller, yet still statistically significant (FIG. 9B; p <0.05). Expression as a percentage of the maximum tension generated by each muscle strip, per entire strip (FIG. 9C) or per weight of the strip (FIG. 9D) revealed no significant differences between sham and OVx groups.

### EXAMPLE 6

### Caveolar Depletion Following OVx

Sarcolemmal caveolae were manually counted in a “blinded” way in 72 coded high magnification photographs (10,000×) obtained from the same 7 pairs of OVx/sham rats. Structures counted as caveolae included all non-coated vesicles present within 100 nanometers (nm) of the myocyte membrane. Counts were only performed in the single myocyte in each high magnification photography displaying an entire membrane perimeter cut in cross-section. These photographs were then scanned (SnapScan310; Agfa) and analyzed using image analysis software (ImagePro I, Media Cybernetics: Silver Spring, Md.). Myocyte profiles were traced manually with a mouse and their length in pixels was determined using this software. Each 1000 pixels of image represented 7.1 micrometers of sarcolemmal length.

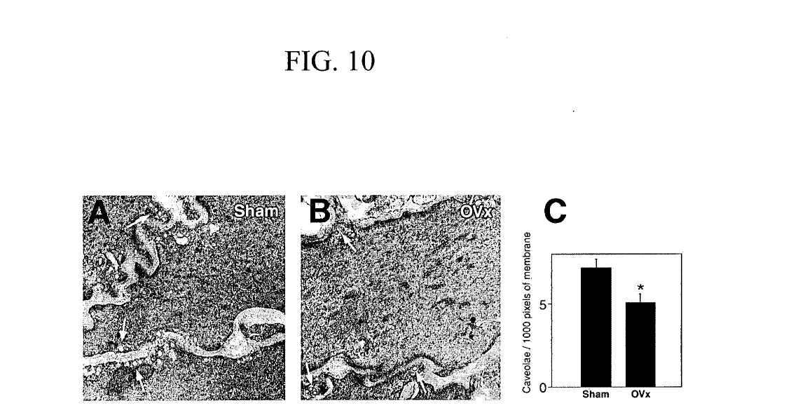

Ultrastructural examination of myocytes in detrusors from sham-operated controls reveled that the sarcolemma displayed the characteristic features of normal mature smooth muscle cells (i.e. alternating thick electron dense bands and much thinner less dense zones, the latter containing rows of uniform sized, flask-shaped surface vesicles-caveolae; FIG. 10A). In contrast, in tissues from OVx animals, the sarcolemmae appeared to be dominated by electron dense band patterns (sometimes in long stretches that covered almost the whole sarcolemma), which were interposed by unevenly spaced thin zones with fewer caveolae (FIG. 10B). Image analysis revealed a 28% decrease in the numbers of caveolae per 1,000 pixels of sarcolemmal length (p<0.005; FIG. 10C).

### EXAMPLE 7

### Caveolin-1 Protein Expression was Down-regulated After OVx

Western blots can be performed as follows. Frozen sample tissue quarters were homogenized in liquid nitrogen. A lysis buffer was used to dissolve the homogenate (25 mM Tris, 150 mM NaCl, 5 mM ethylene diamine tetra-acetic acid (EDTA), 1% TritonX-1000, 0.1% sodium dodecyl sulfate (SDS), plus a protease inhibitor cocktail from Roche, Inc.). After sonication and extracting at 4° C. for 20 minutes, the lysates were centrifuged (10,000 g at 4° C. for 15 minutes) and supernatants were transferred. Following protein quantification using the Bradford method (BIO-RAD), equal amounts of 2×Laemmli buffer (Sigma) was added to each sample, mixed and the samples were snap frozen in small aliquots in liquid nitrogen, and stored at 80° C.

Molecular weight marker (Amersham-Pharmacia) and an equal quantity of protein samples were heat-denatured and then resolved in 10% sodium dodecyl sulfate polyacrylamide gel electrophoresis (SDS-PAGE) followed by transfer to a nitrocellulose membrane (BIO-RAD). For detection of caveolin and actin, the blot was blocked with 5% skim milk in TBST, then cut into two portions at approximately 30 kilodaltons (kD) molecular weight. The upper portion was incubated in monoclonal antibody to α-smooth muscle actin (SIGMA), and the lower one with a polyclonal antibody to caveolin-1 (Transduction Laboratories) at a concentration of 1:1000. Incubation was carried out for 1 hour at room temperature or 4° C. overnight. The blots were washed with TBST 3 times for 10 minutes each time and then incubated with peroxidase labeled anti-mouse (upper part) or anti-rabbit (lower part) secondary antibodies for 1 hour. Following final washes, the blots were developed using the ECL detection system (Amersham). Quantification of Western blot bands of interest was performed using a phosphorimager/densitometry image analysis system (Molecular Dynamics). Quantitative comparisons were made only between bands resolved on the same membrane.

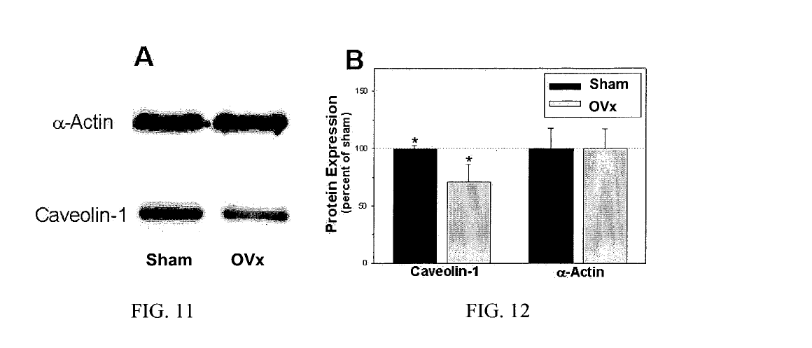

Western blots for caveolin-1 and α-smooth muscle actin were performed using equal amounts of detrusor protein extracts obtained from paired OVx/sham-operated rats. Commassie blue stain was used to ensure both protein integrity and equal loading. A comparison of α-smooth muscles actin signal intensity showed no evident difference between such paired samples. In contrast, there was a modest, but consistent decrease in caveolin-1 expression with OVx in the same blots (FIG. 11). Quantitative analysis using samples from 7 pairs of OVx/sham-operated rats revealed a 30% decrease in caveolin-1 expression after OVx (p<0.005), whereas α-smooth muscle actin expression showed no change (FIG. 12).

### EXAMPLE 8

### Down Regulation of Caveolin- 1 is Reversed by Administration of 17β-estradiol

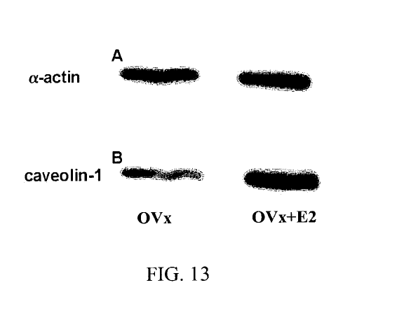

When equal quantities of total protein were examined, the down regulation of caveolin-1 protein 4 months after OVx was effectively reversed by a 1 -month administration of E2. In contrast, α-smooth muscle actin signal intensity showed no evident difference between such paired samples (FIG. 13).

### EXAMPLE 9

### The Relationship between Caveolin-1 and Myocyte Maturation

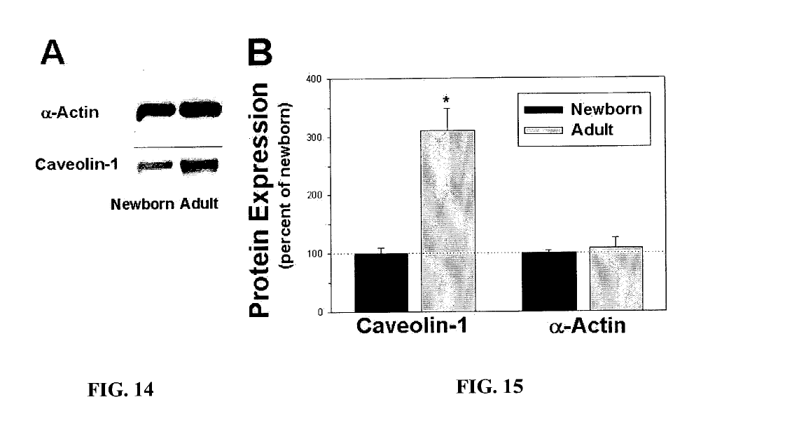

Levels of α-smooth muscle actin were similar in equal quantities of protein extracts obtained from newborn and young adult female rats. In contrast, caveolin-1 protein levels were significantly higher in young adults as compared to newborn animals (FIG. 14). Quantitative analysis using samples from a total of 8 animals revealed a greater than 3-fold increase in caveolin-1 protein in adult, as compared to newborn detrusors (p<0.001), while α-smooth muscle actin expression showed no change (FIG. 15).

### EXAMPLE 10

### 2-D Analysis and Protein Microsequencing

A 2-D gel analysis was then undertaken to identify candidate proteins with different expression levels in OVx and sham-operated rats. For 2-D gel analysis, one quarter of a rat detrusor was homogenized in liquid nitrogen and then resolved in IEF buffer, followed by disruption by sonication and extraction for 20 min at room temperature before centrifugation at 10,000 g at room temperature for 20 min. The protein in the supernatant was quantified and stored at −80° C.

The Mini-PROTEIN II 2-D system (Bio-Rad) was used to resolve protein extracts in two dimensions. Equal amounts (120 pg) of protein from detrusors of sham, OVx, and OVx +E2 treated animals were resolved first through isoelectric focusing (IEF) followed by SDS-PAGE electrophoresis. After electrophoresis, the gels were fixed in 50% methanol for 1 hour and subjected to silver staining.

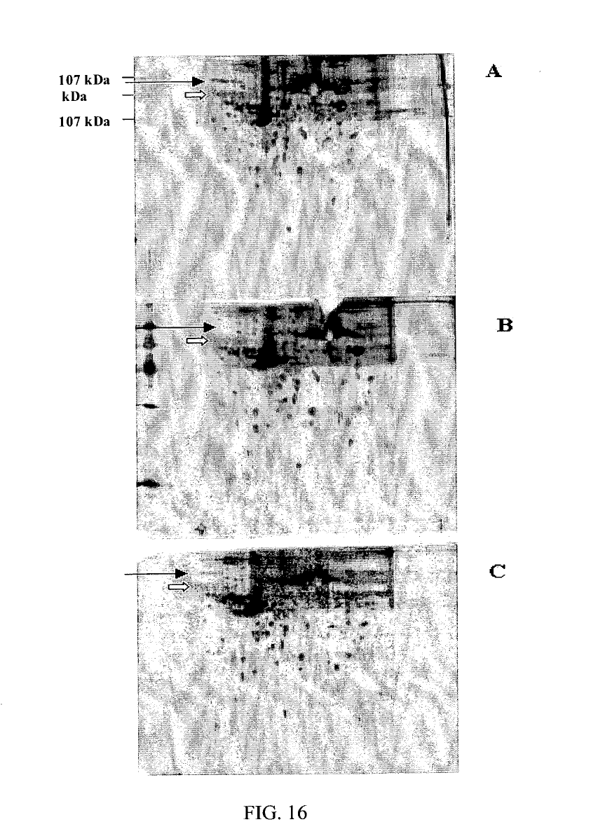

The 2-D gel analysis revealed little difference in protein expression patterns four months after surgery. No proteins appeared to be consistently expressed de novo, while a few proteins were upregulated, and some proteins were shown to be down regulated. Of particular note, a string of protein spots was observed at about 110 kD with an isoelectric point (pI) varying between about 4 to about 5. This string of proteins nearly disappeared four months after OVx, but was restored to approximately normal expression following the subcutaneous implantation of E2 for 1 week (FIG. 16). This protein string was chosen as a candidate for peptide sequencing.

### EXAMPLE 11

### Pooling of the Protein of Interest and Microsequencing

The string of spots at about 100 kD with a pl of 4-5 was excised and processed. A total of nearly 100 spots (about 0.5 pM) was pooled from 30 gels. The chosen spots were isolated with a “neuro punch” (0.65 mm, Fine Science tools, Inc.). Excised spots were aspirated by connecting the neuro punch with a Butterfly-19 needle (Venisystems), and the latter with a 1 ml syringe. An equivalent number of spots was excised from the background to serve as controls, and transferred to a separate tube. Gel slices were then washed twice using 50% HPLC grade acetonitrile in water for 2-3 min. The supernatant was aspirated after each wash, leaving the gel slices moist. Gel spots were accumulated until 0.5-1 picomole of protein was isolated for each sample.

The pooled protein sample was subjected to protein microsequencing analysis by microcapillary reverse phase HPLC nano-electrospray tandem mass spectrometry (μLC/MS/MS) on a Finnigan LQC quadrupole ion trap mass spectrometer. The following sequences resulted from microsequencing of the pooled extract:

| | |
|---|---|
| SQNEQDTVLDGDFIVR | SEQ ID NO: 1 |
| IPAQGGTNINK | SEQ ID NO: 2 |
| ITNIIPDEYFNR | SEQ ID NO: 13 |
| NNQIDHIDEK | SEQ ID NO: 14 |
| EEAVSASLK | SEQ ID NO: 15 |
| SKAEAESLYQSK | SEQ ID NO: 16 |
| TLNDMRQEYEQLIAK | SEQ ID NO: 17 |

Database searching such as PubMed of NCBI was used to identify the proteins that correspond to the peptides isolated. The protein of interest was further confirmed by immunoblotting and other biochemical approaches.

### EXAMPLE 12

### Characterization of SEQ ID NOs: 13-15

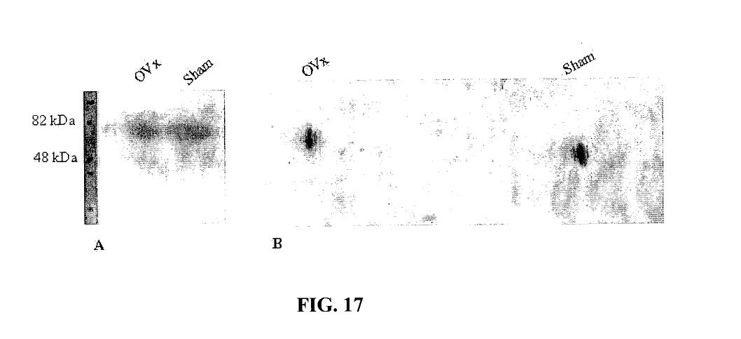

Database and literature searching revealed that the peptides corresponding to SEQ ID NOs: 13-15 correspond to a protein called Lumican. Lumican is a member of the small intestinal proteoglycan proteins with leucine-rich repeat (LRR) motifs, and is widely expressed in the extracellular matrix of many tissues. Western blotting combined with protein resolution in 1 -D or 2-D gels revealed no difference between OVx and sham controls, but a “smeared” signal was observed in 2-D gels at about 80 kD with vertically long streaks (FIG. 17). Matching of the smeared streaks with the spot of interest placed Lumican below the spots of interest. Lumican was thus provisionally excluded as the protein of interest.

### EXAMPLE 13

### Characterization of SEQ ID NOs: 1 and 2

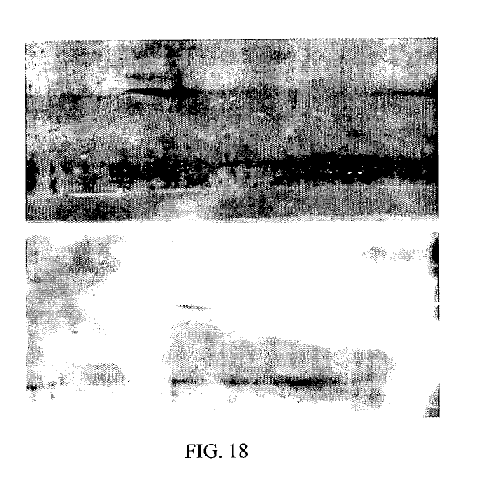

Database and literature searching revealed that SEQ ID NOs: 1 and 2 correspond to a protein in the ITI-H4 family. It is thus hypothesized that SEQ ID NOs: 1 and 2 correspond to DERP. Combining 2-D gel electrophoresis with Western blot analysis using antiserum raised against pig ITI-H4 reveals a clear signal at about 110 kD and a pI of 4-5 (FIG. 18). In addition, signals from the sham samples were stronger than those from the OVx group. It is estimated that the level of DERP in the OVx group is 10% or less of that in the sham control group. The DNA sequence of rat ITI-H4 is SEQ ID NO: 3 (accession number NM_{—}2292987 or gi. Y11283). The DNA sequence of human ITI-H4 is SEQ ID NO: 5 (accession number NM_{—}002218 or gi. 4504784).

### EXAMPLE 14

### DERP is Present in Cultured Rat Smooth Muscle Cells

Polyclonal antibodies to SEQ ID NOs: 1 and 2 were made in rabbits by techniques known in the art. The two antibodies to the rat peptides are called anti-DERP-1 and anti-DERP-2. In addition, two polyclonal antibodies have been made to human ITI-H4 using peptides SEQ ID NO: 18 (ANTVQEATFQMELPKK) and SEQ ID NO: 19 (RVQGNDHSATRERRLD). The antibodies to the human DERP peptides are called anti-DERP-3 and anti-DERP-4, respectively.

Primary bladder smooth muscle cell cultures were established using neonatal Sprague Dawley rat pups. After sacrifice, bladders were removed and immediately placed in cold Earle's balanced salt solution (EBSS). In a culture hood, bladders were minced to 1 mm cubes, and then transferred to a dissociation solution of EBSS containing 0.5 mg/ml collagenase (Worthington Biochemical Corporation), 0.5 mg/ml elastase (Worthington Biochemical Corporation), 1 mg/ml soy trypsin inhibitor (Invitrogen) and 2 μg/ml DNAse (Worthington Biochemical Corporation). Digestion was carried out at 37° C., with trituration 40 times through the fire polished tip of a long Pasteur pipette at 10-15 minute intervals until tissue acquired a torn tissue paper-like appearance. This suspension was passed through a 40 micron strainer to remove the undigested uroepithelial layer. Cells were pelleted by centrifugation for 4 minutes at 1000 rpm and then resuspended in plating medium consisting of DMEM/F12 supplemented with 1% serum. Cells were plated overnight on Falcon 8 well culture slides (Fisher) coated with human placental collagen IV (10 mg/cm²) and laminin from mouse sarcoma (2 μg/cm²). The following day cells were switched to serum free medium DMEM/F12 (Invitrogen) with N2 supplement (Invitrogen) and 100 U/ml penicillin and 100 μg/ml streptomycin. Cultures were fed by replacing half the medium every second day and maintained in a humidified incubator at 37° C. with 5% CO₂ for 4-7 days prior to treatment with 100 nM of 17-β-estradiol (E2; Sigma), 17-β-estradiol (Sigma) or E2 with ICI-182, 780 (Tocris Cookson) for 24 hours. Cells were fixed with 4% paraforrnaldehyde prior to immunohistochemical studies.

Double label immunocytochemistry was performed using a monoclonal antibody to α-smooth muscle actin (SIGMA) (1:5000), and a polyclonal antibody to DERP (anti-DERP-1)(1:1000). The DERP polyclonal antibody was visualized with Alexa 488-tagged goat anti rabbit F(ab) immunoglobulin G (H+L) (Molecular Probes) and the α-smooth muscle actin monoclonal antibodies were visualized with CY3-tagged donkey anti-mouse IgG (H+L) (Jackson Immunoresearch Laboratories). Cells were viewed and confocal images obtained using a Zeiss LSM 410 confocal microscope.

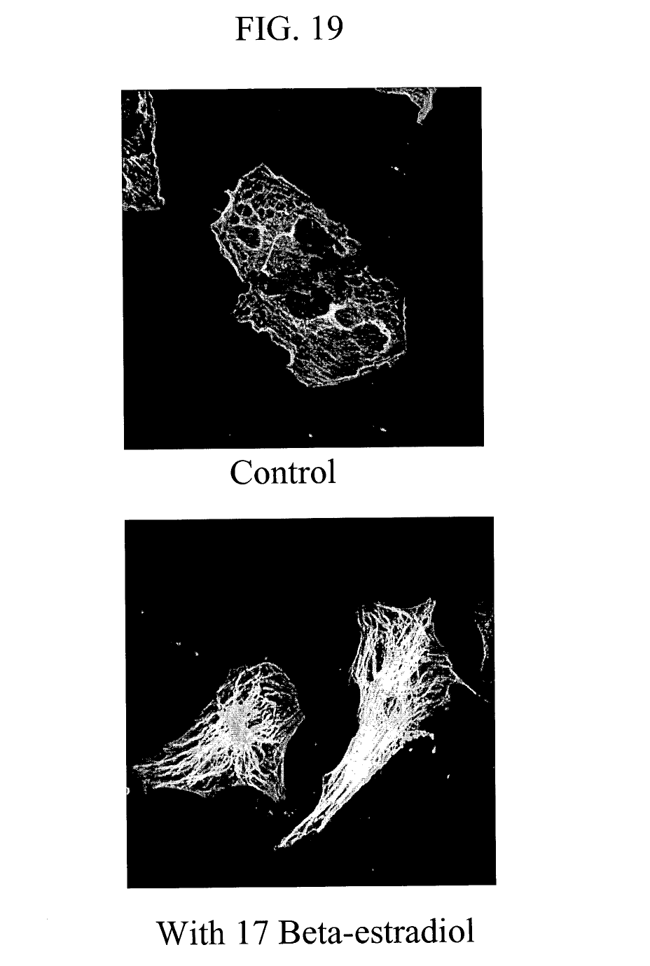

The red staining is for smooth muscle actin and the green is for DERP, where they overlap it is yellow (FIG. 19). The addition of estrogen to cultured smooth muscle cells clearly enhances DERP protein reactivity.

### EXAMPLE 15

### Effect of Long Term OVx on Serum DERP Expression in the Rat

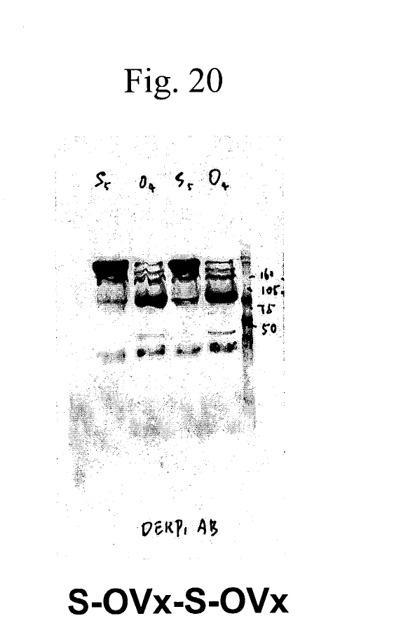

FIG. 20 is a Western blot using the anti-DERP-1 antibody. It shows expression of a large band in sham (S) samples which is not present in ovariectomized rats (OVx). The band seems to migrate more slowly (140-160 kD instead of the expected 120 kD) than DERP but that could be due to extensive glycosylation. There is also the appearance of a stronger smaller band in OVx samples. The smaller band could be due to cleavage of DERP. It turns out that ITI-H4 is highly vulnerable to kallikrein cleavage at one site. Thus there are two alternative hypotheses to why serum DERP goes down with OVx: either there may be decreased DERP synthesis and/or there may be increased kallikrein mediated cleavage. ITI-H4 is known to have a kallikrein sensitive cleavage site which would result in two fragments whose predicted size (about 80 and 40 kD) resembles those seen in this blot (Salier et al. Biochem. J. 315, (Pt. 1) 1-9 (1996)). Thus, it is possible that the down-regulation of DERP after OVx may be, at least in part, mediated by kallikrein cleavage of this protein into two protein fragments.

### EXAMPLE 16

### Probing with Primers to DERP mRNA

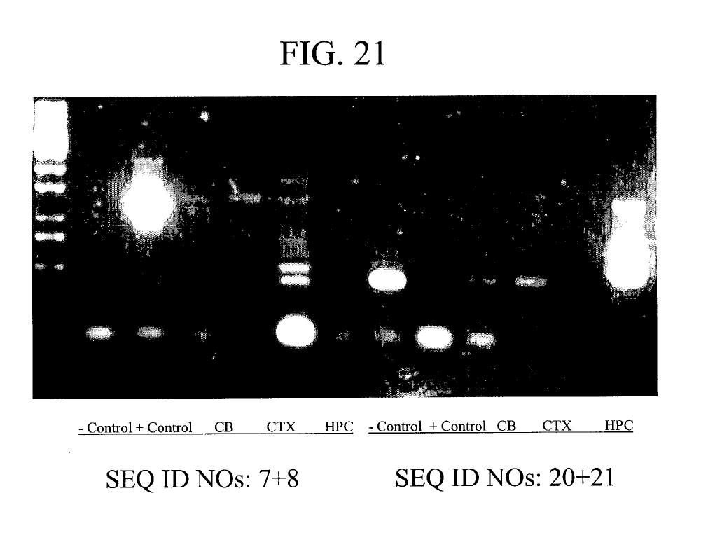

Primers corresponding to SEQ ID NOs: 7and 8 were used in RT PCR reactions to probe for DERP MRNA (FIG. 21, Primer CT1).

| | |
|---|---|
| 5′ GGATCCAGTGGGCAGATGCACCTGC | SEQ ID NO: 7 |
| 5′ GGATCCCTATATCTTCACCGTCCAGC | SEQ ID NO: 8 |

The arrow shows the DERP product. As is shown, the DERP product is observed in the positive control (liver tissue) and in the cortex and hippocampus. No product is seen in the cerebellum which is consistent with the hypothesis that there are no estrogen receptors in the brain.

The RT-PCR reaction was repeated with primers corresponding to SEQ ID NOs:20 and 21.

| | |
|---|---|
| 5′ GCAACGGAAGTCTCAGAATGAG | SEQ ID NO: 20 |
| 5′ GCTTTATTGATGTTGGTCCCTC | SEQ ID NO: 21 |

The arrow shows the DERP product. As is shown, the DERP product is observed in the positive control (liver tissue) and in the cortex and hippocampus.

Other DERP primers pairs are shown below

| | |
|---|---|
| 5′ CTTCCCGA TTTGCTCATACTG | SEQ ID NO: 22 |
| 5′ GGGCTCAAAGATGTAGATGTC | SEQ ID NO: 23 |
| 5′ AGGGACCAACATCAATAAAGC | SEQ ID NO: 24 |
| 5′ TTTGGCTAAGAGGACATCAG | SEQ ID NO: 25 |
| 5′ TAGACAATACATGCCTCCTC | SEQ ID NO: 26 |
| 5′ GTCACCACCAGTCGTTCAG | SEQ ID NO: 27 |
| 5′ GGAGGTGGTTGGCAAGTATG | SEQ ID NO: 28 |
| 5′ CGTCCAGCAGGAAATCTC | SEQ ID NO: 29 |
| 5′ CAGACATTGTCCAGACTCGG | SEQ ID NO: 30 |
| 5′ AGCCAGGTAACACAGAGAACAG | SEQ ID NO: 31 |
| 5′ ACAATACATGCCTCCTCC | SEQ ID NO: 32 |
| 5′ ATATCCACACAGAGCTGG | SEQ ID NO: 33 |
| 5′ TCTCAGAATGAGCAGGACACGG | SEQ ID NO: 34 |
| 5′ CAGGCAGAAGAGGCTATACCGC | SEQ ID NO: 35 |
| 5′ TTCAAGCCGACACTCTCC | SEQ ID NO: 36 |
| 5′ TTTATTGATGTTGGTCCCTC | SEQ ID NO: 37 |
| 5′ GGATCCCTCTCAGCAGTTCAACGGC | SEQ ID NO: 38 |
| 5′ GGATCCCTATATCTTCACCGTCCAGC | SEQ ID NO: 39 |
| 5′ GGATCCTTAGCCAAAGTCAGTGGGCAG | SEQ ID NO: 40 |
| 5′ GGATCCCTATATCTTCACCGTCCAGC | SEQ ID NO: 41 |
| 5′ GGATCCGGAGCTGAGTTAGAGGCCCTCG | SEQ ID NO: 42 |
| 5′ GGATCCCTATATCTTCACCGTCCAGC | SEQ ID NO: 43 |
| 5′ CGCGGATCCGCG TTTGCTTCTAGCATTGAC | SEQ ID NO: 44 |
| 5′ CCGGAATTCCGG CTATATCTTCACCGTC | SEQ ID NO: 45 |
| 5′ CGCGGATCCGCG GACCAGCTCTGTGTGGATAT | SEQ ID NO: 46 |
| 5′ CCGGAATTCCGG CTATATCTTCACCGTC | SEQ ID NO: 45 |
| 5′ CGCGGATCCGCG AAAGTGGTGGAACAAGA | SEQ ID NO: 47 |
| 5′ CCGGAATTCCGG CTATATCTTCACCGTC | SEQ ID NO: 45 |

A protein related to related to bladder dysfunction has been discovered and named DERP for Detrusor Estrogen-Regulated protein. Immunologically active fragments of DERP have been used to make anti-DERP antibodies which are useful to detect levels of DERP in cells and in biological fluids. Anti-DERP antibodies may be used in methods of diagnosing DERP-related conditions such as, for example, detrusor hyperactivity and urinary incontinence. Detection of DERP levels may be used to determine if estrogen therapy is indicated as a treatment for bladder dysfunction and other related conditions.

While the invention has been described with reference to a preferred embodiment, it will be understood by those skilled in the art that various changes may be made and equivalents may be substituted for elements thereof without departing from the scope of the invention. In addition, many modifications may be made to adapt a particular situation or material to the teachings of the invention without departing from essential scope thereof. Therefore, it is intended that the invention not be limited to the particular embodiment disclosed as the best mode contemplated for carrying out this invention, but that the invention will include all embodiments falling within the scope of the appended claims.

All cited patents, patent applications, and other references are incorporated herein by reference in their entirety.

What is claimed is:

1. A purified immunogenic polypeptide fragment, the amino acid sequence of which comprises about ten to about fifty consecutive amino acids of SEQ ID NO: 4 or SEQ ID NO: 6.

2. The purified immunogenic polypeptide of claim 1, comprising fifteen to thirty consecutive amino acids of SEQ ID NO: 4 or SEQ ID NO: 6.

3. The purified immunogenic polypeptide of claim 1, comprising twenty to twenty-five consecutive amino acids of SEQ ID NO: 4 or SEQ ID NO: 6.

4. The purified immunogenic polypeptide of claim 1, wherein the polypeptide is SEQ ID NO: 1, SEQ ID NO:2, SEQ ID NO: 18, SEQ ID NO: 19, or a mixture comprising one or more of the foregoing polypeptides.

5. An isolated antibody immunochemically reactive with an immunogenic polypeptide fragment comprising about fifteen to about fifty amino acids of SEQ ID NO: 4 or SEQ ID NO: 6.

6. The isolated antibody of claim 5 wherein the polypeptide fragment is SEQ ID NO: 1, SEQ ID NO: 2, SEQ ID NO: 18, or SEQ ID NO: 19.

7. The isolated antibody of claim 5, comprising a polyclonal antibody.

8. The isolated antibody of claim 5, wherein the antibody is labeled with an enzyme, a biotin, a fluorophore, a chromophore, a heavy metal, a paramagnetic isotope, or a radioisotope

9. A method of detecting a DERP polypeptide in a biological sample, comprising contacting the biological sample with an antibody according to claim 5 and detecting any DERP polypeptide-antibody complexes formed.

10. The method of claim 9, wherein detecting is by immunoenzymatic assay.

11. The method of claim 9, wherein the biological sample is a cell.

12. The method of claim 9, wherein the biological sample is a fluid sample.

13. The method of claim 12, wherein the fluid sample is a tissue extract, urine, blood, serum, central nervous system fluid, or phlegm.

14. The method of claim 9, further comprising:
comparing a level of DERP polypeptide-anti-DERP antibody complexes in the biological sample with a level of DERP polypeptide-anti-DERP antibody complexes in a reference sample, and
diagnosing a DERP-related disorder when the level of DERP polypeptide-anti-DERP antibody complexes in the biological sample is about 25% or less of the level of DERP polypeptide-anti-DERP antibody complex in the reference sample, wherein the biological sample is a sample from a mammal.

15. The method of claim 14, wherein the DERP-related disorder is impaired detrusor contractility, urinary retention, Alzheimer's disease, cardiovascular disease, osteoporosis, or a combination comprising one or more of the foregoing disorders.

16. The method of claim 14, wherein the biological sample is a fluid sample.

17. The method of claim 16 wherein the fluid sample is a tissue extract, urine, blood, serum, central nervous system fluid, or phlegm.

18. The method of claim 14, wherein diagnosing a DERP-related disorder confirms an estrogen-related disorder.

19. The method of claim 18, further comprising administering to the mammal a therapeutically effective amount of estrogen in a pharmaceutically effective carrier.

20. The method of claim 14, wherein the diagnosis of a DERP-related disorder confirms the presence of impaired detrusor contractility.

21. The method of claim 20, further comprising administering to the mammal a therapeutically effective amount of an antispasmodic drug.

22. A kit for the detection of DERP polypeptide in a biological sample, comprising:
an anti-DERP antibody;
a first reagent for preparation of a medium suitable for carrying out an immunological reaction;
a second reagent for the detection of DERP polypeptide-anti-DERP antibody complexes formed during an immunological reaction; and
a reference sample comprising a known quantity of a DERP polypeptide.

23. A method of detecting a DERP mRNA comprising:
contacting a sample with a first primer and a second primer each primer comprising greater than or equal to about ten nucleotides of SEQ ID NO: 5 or SEQ ID NO: 6,
performing a reverse transcription and polymerase chain reaction; and
detecting the reaction product;
wherein detecting a reaction product confirms the presence of the DERP mRNA.

24. The method of claim 23, wherein the first primer is SEQ ID NO: 7, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 44, SEQ ID NO: 46, or SEQ ID NO: 47;
and the second primer is SEQ ID NO: 8, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41, SEQ ID NO: 43, or SEQ ID NO: 45.
